(19) United States
(12) Patent Application Publication
(10) Pub. No.: US 2026/0028341 A1
(43) Pub. Date: Jan. 29, 2026

(54) IMIDAZOTRIAZINE DERIVATIVES AS IL-17 MODULATORS

(71) Applicant: UCB BIOPHARMA SRL, Brusels (BE)

(72) Inventors: Gareth Neil BRACE, Oxfordshire (GB); Paul GOLDSMITH, Slough, Berkshire (GB); Gregory William HASLETT, Slough, Berkshire (GB); Timothy John norman, Slough, Berkshire (GB); Robert STRAKER, Slough, Berkshire (GB); Richard TAYLOR, Slough, Berkshire (GB); Robert James TOWNSEND, Oxfordshire (GB); Giancarlo TRANI, Oxfordshire (GB)

(21) Appl. No.: 18/994,318

(22) PCT Filed: Jul. 18, 2023
(86) PCT No.: PCT/EP2023/069895

§ 371 Date: Jan. 14, 2025

(30) Foreign Application Priority Data
Jul. 22, 2022 (GB) 2210731.2

(51) Int. Cl. C07D 487/04; A61K 31/53

(52) U.S. Cl. CPC C07D 487/04; A61K 31/53

(57) ABSTRACT

A series of substituted imidazo[1,2-b][1,2,4]triazine derivatives of Formula (I) as defined herein, being potent modulators of human IL-17 activity, are accordingly of benefit in the treatment and/or prevention of various human ailments, including inflammatory and autoimmune disorders.

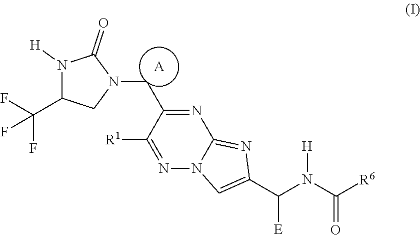

The present invention relates to heterocyclic compounds, and to their use in therapy. More particularly, this invention is concerned with pharmacologically active substituted imidazo[1,2-b][1,2,4]triazine derivatives. These compounds act as modulators of IL-17 activity, and are accordingly of benefit as pharmaceutical agents for the treatment and/or prevention of pathological conditions, including adverse inflammatory and autoimmune disorders.

IL-17A (originally named CTLA-8 and also known as IL-17) is a pro-inflammatory cytokine and the founder member of the IL-17 family (Rouvier et al., J Immunol., 1993, 150, 5445-5456). Subsequently, five additional members of the family (IL-17B to IL-17F) have been identified, including the most closely related, IL-17F (ML-1), which shares approximately 55% amino acid sequence homology with IL-17A (Moseley et al., Cytokine Growth Factor Rev., 2003, 14, 155-174). IL-17A and IL-17F are expressed by the recently defined autoimmune related subset of T helper cells, Th17, that also express IL-21 and IL-22 signature cytokines (Kom et al., Ann. Rev. Immunol., 2009, 27, 485-517). IL-17A and IL-17F are expressed as homodimers, but may also be expressed as the IL-17A/F heterodimer (Wright et al., J. Immunol., 2008, 181, 2799-2805). IL-17A and F signal through the receptors IL-17R, IL-17RC or an IL-17RA/RC receptor complex (Gaffen, Cytokine, 2008, 43, 402-407). Both IL-17A and IL-17F have been associated with a number of autoimmune diseases.

The compounds in accordance with the present invention, being potent modulators of human IL-17 activity, are therefore beneficial in the treatment and/or prevention of various human ailments, including inflammatory and autoimmune disorders.

Furthermore, the compounds in accordance with the present invention may be beneficial as pharmacological standards for use in the development of new biological tests and in the search for new pharmacological agents. Thus, the compounds of this invention may be useful as radioligands in assays for detecting pharmacologically active compounds.

WO 2013/116682 and WO 2014/066726 relate to separate classes of chemical compounds that are stated to modulate the activity of IL-17 and to be useful in the treatment of medical conditions, including inflammatory diseases.

WO 2018/229079 and WO 2020/011731 describe spirocyclic molecules that are stated to act as modulators of IL-17 activity, and thus to be of benefit in the treatment of pathological conditions including adverse inflammatory and autoimmune disorders.

WO 2019/138017, WO 2020/260425, WO 2020/260426, WO 2020/261141, WO 2021/170627, WO 2021/170631, WO 2021/204800, WO 2021/204801, WO 2022/096411, WO 2022/096412 and WO 2022/128584 describe various classes of fused bicyclic imidazole derivatives that are stated to act as modulators of IL-17 activity and thus to be of benefit in the treatment of pathological conditions including adverse inflammatory and autoimmune disorders. Fused bicyclic imidazole derivatives operating as modulators of IL-17 activity are also described in co-pending international patent application PCT/EP2022/068165 (published on 5 Jan. 2023 as WO 2023/275301).

WO 2020/120140 and WO 2020/120141 describe discrete classes of chemical compounds that are stated to act as modulators of IL-17 activity, and thus to be of benefit in the treatment of pathological conditions including adverse inflammatory and autoimmune disorders.

Heterocyclic compounds that are stated to inhibit IL-17A and to be useful as immunomodulators are described in WO 2019/223718, WO 2021/027721, WO 2021/027722, WO 2021/027724, WO 2021/027729 and WO 2021/098844.

Heterocyclic compounds stated to be capable of modulating IL-17 activity are also described in WO 2020/127685, WO 2020/146194 and WO 2020/182666.

None of the prior art available to date, however, discloses or suggests the precise structural class of substituted imidazo[1,2-b][1,2,4]triazine derivatives as provided by the present invention.

As well as being potent modulators of human IL-17 activity, the compounds in accordance with the present invention possess other notable advantages. In particular, the compounds of the invention display valuable metabolic stability, as determined in either microsomal or hepatocyte incubations. The compounds of the invention also display valuable permeability as determined by standard assays, e.g. the Caco-2 permeability assay.

The present invention provides a compound of formula (I) or an N-oxide thereof, or a pharmaceutically acceptable salt thereof:

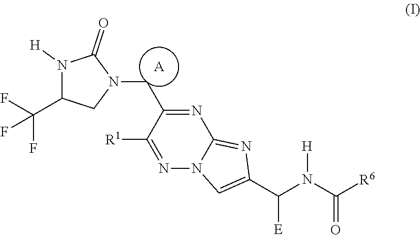

wherein
E represents a group of formula (Ea), (Eb), (Ec), (Ed) or (Ee):

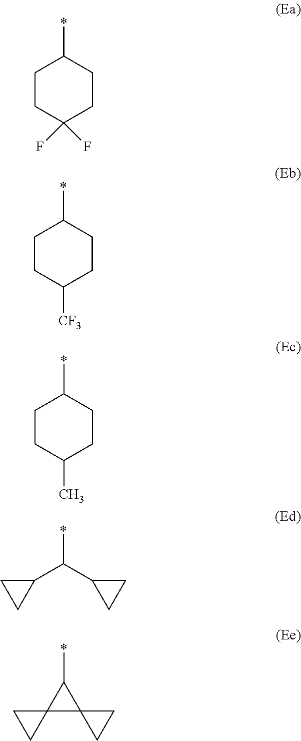

in which the asterisk (*) represents the point of attachment to the remainder of the molecule;
ring A represents C₃₋₇ cycloalkyl or C₃₋₇ heterocycloalkyl, either of which groups may be optionally substituted by one or more substituents;
R¹ represents hydrogen, fluoro, chloro, methyl, difluoromethyl or trifluoromethyl;
R⁶ represents —OR^{6a} or —NR^{6b}R⁶; or R⁶ represents C₁₋₆ alkyl, C₃₋₉ cycloalkyl, C₃₋₉ cycloalkyl(C₁₋₆)alkyl, aryl, aryl(C₁₋₆)alkyl, C₃₋₇ heterocycloalkyl, C₃₋₇ heterocycloalkyl-(C₁₋₆)alkyl, heteroaryl or heteroaryl(C₁₋₆)alkyl, any of which groups may be optionally substituted by one or more substituents;
R^{6a} represents C₁₋₆ alkyl; or R^{6a} represents C₃₋₉ cycloalkyl or C₃₋₇ heterocycloalkyl, either of which groups may be optionally substituted by one or more substituents;
R^{6b} represents hydrogen or C₁₋₆ alkyl; and
R^{6c} represents hydrogen or C₁₋₆ alkyl; or
R^{6b} and R^{6c}, when taken together with the nitrogen atom to which they are both attached, represent azetidin-1-yl, pyrrolidin-1-yl, oxazolidin-3-yl, isoxazolidin-2-yl, thiazolidin-3-yl, isothiazolidin-2-yl, piperidin-1-yl, morpholin-4-yl, thiomorpholin-4-yl, piperazin-1-yl, homopiperidin-1-yl, homomorpholin-4-yl or homopiperazin-1-yl, any of which groups may be optionally substituted by one or more substituents.

The present invention also provides a compound of formula (I) as defined above, or a pharmaceutically acceptable salt thereof.

The present invention also provides a compound of formula (I) as defined above or an N-oxide thereof, or a pharmaceutically acceptable salt thereof, for use in therapy.

The present invention also provides a compound of formula (I) as defined above or an N-oxide thereof, or a pharmaceutically acceptable salt thereof, for use in the treatment and/or prevention of disorders for which the administration of a modulator of IL-17 function is indicated.

The present invention also provides the use of a compound of formula (I) as defined above or an N-oxide thereof, or a pharmaceutically acceptable salt thereof, for the manufacture of a medicament for the treatment and/or prevention of disorders for which the administration of a modulator of IL-17 function is indicated.

The present invention also provides a method for the treatment and/or prevention of disorders for which the administration of a modulator of IL-17 function is indicated which comprises administering to a patient in need of such treatment an effective amount of a compound of formula (I) as defined above or an N-oxide thereof, or a pharmaceutically acceptable salt thereof.

Where any of the groups in the compounds of formula (I) above is stated to be optionally substituted, this group may be unsubstituted, or substituted by one or more substituents. Generally, such groups will be unsubstituted, or substituted by one, two, three or four substituents. Typically, such groups will be unsubstituted, or substituted by one, two or three substituents. Suitably, such groups will be unsubstituted, or substituted by one or two substituents.

For use in medicine, the salts of the compounds of formula (I) will be pharmaceutically acceptable salts. Other salts may, however, be useful in the preparation of the compounds of formula (I) or of their pharmaceutically acceptable salts. Standard principles underlying the selection and preparation of pharmaceutically acceptable salts are described, for example, in Handbook of Pharmaceutical Salts: Properties, Selection and Use, ed. P. H. Stahl & C. G. Wermuth, Wiley-VCH, 2002. Suitable pharmaceutically acceptable salts of the compounds of formula (I) include acid addition salts which may, for example, be formed by mixing a solution of a compound of formula (I) with a solution of a pharmaceutically acceptable acid.

The present invention also includes within its scope co-crystals of the compounds of formula (I) above. The technical term “co-crystal” is used to describe the situation where neutral molecular components are present within a crystalline compound in a definite stoichiometric ratio. The preparation of pharmaceutical co-crystals enables modifications to be made to the crystalline form of an active pharmaceutical ingredient, which in turn can alter its physicochemical properties without compromising its intended biological activity (see Pharmaceutical Salts and Co-crystals, ed. J. Wouters & L. Quere, RSC Publishing, 2012).

Suitable alkyl groups which may be present on the compounds of use in the invention include straight-chained and branched C₁₋₆ alkyl groups, for example C₁₋₄ alkyl groups. Typical examples include methyl and ethyl groups, and straight-chained or branched propyl, butyl and pentyl groups. Particular alkyl groups include methyl, ethyl, n-propyl, isopropyl, n-butyl, sec-butyl, isobutyl, tert-butyl, 2,2-dimethylpropyl and 3-methylbutyl. Derived expressions such as “C₁₋₆ alkoxy”, “C₁₋₆ alkylthio”, “C₁₋₆ alkylsulphonyl” and “C₁₋₆ alkylamino” are to be construed accordingly.

The term “C₃₋₉ cycloalkyl” as used herein refers to monovalent groups of 3 to 9 carbon atoms derived from a saturated monocyclic hydrocarbon, and may comprise benzo-fused analogues thereof. Suitable C₃₋₉ cycloalkyl groups include cyclopropyl, cyclobutyl, benzocyclobutenyl, cyclopentyl, indanyl, cyclohexyl, cycloheptyl, cyclooctyl and cyclononanyl.

The term “aryl” as used herein refers to monovalent carbocyclic aromatic groups derived from a single aromatic ring or multiple condensed aromatic rings. Suitable aryl groups include phenyl and naphthyl, preferably phenyl.

Suitable aryl(C₁₋₆)alkyl groups include benzyl, phenylethyl, phenylpropyl and naphthylmethyl.

The term “C₃₋₇ heterocycloalkyl” as used herein refers to saturated monocyclic rings containing 3 to 7 carbon atoms and at least one heteroatom selected from oxygen, sulphur and nitrogen, and may comprise benzo-fused analogues thereof. Suitable heterocycloalkyl groups include oxetanyl, azetidinyl, tetrahydrofuranyl, dihydrobenzo-furanyl, dihydrobenzothienyl, pyrrolidinyl, indolinyl, isoindolinyl, oxazolidinyl, thiazolidinyl, isothiazolidinyl, imidazolidinyl, tetrahydropyranyl, chromanyl, tetrahydro-thiopyranyl, piperidinyl, 1,2,3,4-tetrahydroquinolinyl, 1,2,3,4-tetrahydroisoquinolinyl, piperazinyl, 1,2,3,4-tetrahydroquinoxalinyl, hexahydro-[1,2,5]thiadiazolo[2,3-a]-pyrazinyl, homopiperazinyl, morpholinyl, benzoxazinyl, thiomorpholinyl, azepanyl, oxazepanyl, diazepanyl, thiadiazepanyl and azocanyl.

The term “heteroaryl” as used herein refers to monovalent aromatic groups containing at least 5 atoms derived from a single ring or multiple condensed rings, wherein one or more carbon atoms have been replaced by one or more heteroatoms selected from oxygen, sulphur and nitrogen. Suitable heteroaryl groups include furyl, benzofuryl, dibenzofuryl, thienyl, benzothienyl, thieno[2,3-c]pyrazolyl, thieno[3,4-b]-[1,4]dioxinyl, dibenzothienyl, pyrrolyl, indolyl, pyrrolo[2,3-b]pyridinyl, pyrrolo[3,2-c]-pyridinyl, pyrrolo[3,4-b]pyridinyl, pyrazolyl, pyrazolo[1,5-a]pyridinyl, 4,5,6,7-tetrahydropyrazolo[1,5-a]pyridinyl, pyrazolo[3,4-d]pyrimidinyl, pyrazolo[1,5-a]-pyrazinyl, indazolyl, 4,5,6,7-tetrahydroindazolyl, oxazolyl, benzoxazolyl, isoxazolyl, thiazolyl, benzothiazolyl, isothiazolyl, imidazolyl, benzimidazolyl, imidazo[2,1-b]-thiazolyl, imidazo[1,2-a]pyridinyl, 5,6,7,8-tetrahydroimidazo[1,2-a]pyridinyl, imidazo-[4,5-b]pyridinyl, imidazo[1,2-b]pyridazinyl, purinyl, imidazo[1,2-a]pyrimidinyl, imidazo-[1,2-c]pyrimidinyl, imidazo[1,2-a]pyrazinyl, oxadiazolyl, thiadiazolyl, triazolyl, [1,2,4]-triazolo[1,5-a]pyridinyl, [1,2,4]triazolo[4,3-a]pyridinyl, [1,2,4]triazolo[4,3-a]pyrazinyl, 5,6,7,8-tetrahydro[1,2,4]triazolo[4,3-a]pyridinyl, [1,2,4]triazolo[1,5-a]pyrimidinyl, 6,8-dihydro-5H-[1,2,4]triazolo[4,3-a]pyrazinyl, benzotriazolyl, tetrazolyl, pyridinyl, quinolinyl, isoquinolinyl, naphthyridinyl, pyridazinyl, cinnolinyl, phthalazinyl, pyrimidinyl, quinazolinyl, pyrazinyl, quinoxalinyl, pteridinyl, triazinyl and chromenyl groups.

The term “halogen” as used herein is intended to include fluorine, chlorine, bromine and iodine atoms, typically fluorine, chlorine or bromine.

Where the compounds of formula (I) have one or more asymmetric centres, they may accordingly exist as enantiomers. Where the compounds in accordance with the invention possess two or more asymmetric centres, they may additionally exist as diastereomers. The invention is to be understood to extend to the use of all such enantiomers and diastereomers, and to mixtures thereof in any proportion, including racemates. Formula (I) and the formulae depicted hereinafter are intended to represent all individual stereoisomers and all possible mixtures thereof, unless stated or shown otherwise. In addition, compounds of formula (I) may exist as tautomers, for example keto (CH₂C═O)↔enol (CH═CHOH) tautomers or amide (NHC═O)↔hydroxyimine (N═COH) tautomers. Formula (I) and the formulae depicted hereinafter are intended to represent all individual tautomers and all possible mixtures thereof, unless stated or shown otherwise.

It is to be understood that each individual atom present in formula (I), or in the formulae depicted hereinafter, may in fact be present in the form of any of its naturally occurring isotopes, with the most abundant isotope(s) being preferred. Thus, by way of example, each individual hydrogen atom present in formula (I), or in the formulae depicted hereinafter, may be present as a ¹H, ²H (deuterium) or ³H (tritium) atom, preferably ¹H. Similarly, by way of example, each individual carbon atom present in formula (I), or in the formulae depicted hereinafter, may be present as a ¹²C, ¹³C or ¹⁴C atom, preferably ¹²C.

In a first embodiment, E represents a group of formula (Ea). In a second embodiment, E represents a group of formula (Eb). In a third embodiment, E represents a group of formula (Ec). In a fourth embodiment, E represents a group of formula (Ed). In a fifth embodiment, E represents a group of formula (Ee).

Suitably, E represents a group of formula (Ea) or (Ed), especially (Ea).

Generally, the present invention provides a compound of formula (IA-1), (IA-2), (IA-3), (IA-4) or (IA-5) or an N-oxide thereof, or a pharmaceutically acceptable salt thereof:

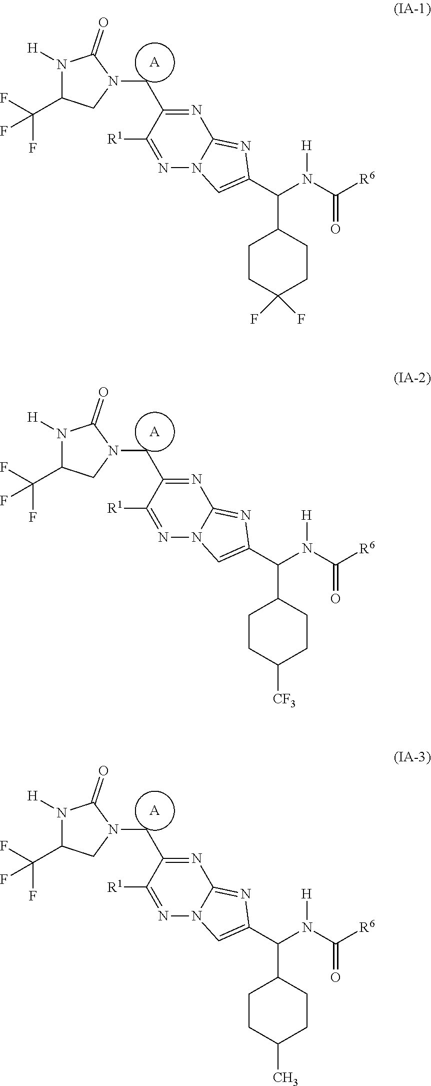

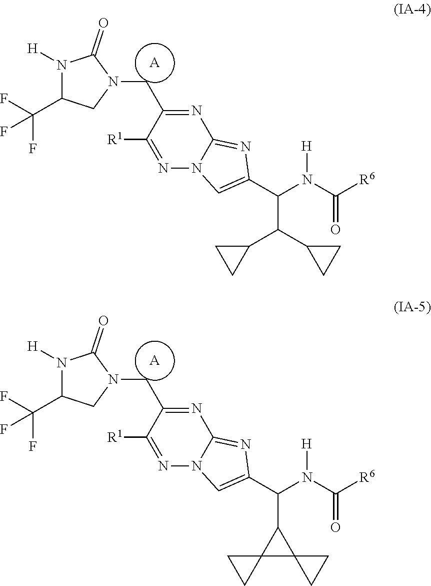

wherein A, R¹ and R⁶ are as defined above.

Typically, the present invention provides a compound of formula (IA-1) or (IA-4) as defined above or an N-oxide thereof, or a pharmaceutically acceptable salt thereof.

Suitably, the present invention provides a compound of formula (IA-1) as defined above or an N-oxide thereof, or a pharmaceutically acceptable salt thereof.

Generally, ring A represents C₃₋₇ cycloalkyl or C₃₋₇ heterocycloalkyl, either of which groups may be unsubstituted, or substituted by one or more substituents, typically by one or two substituents. Suitably, ring A may represent C₃₋₆ cycloalkyl or C₄₋₆ heterocycloalkyl, either of which groups may be unsubstituted, or substituted by one or more substituents, typically by one or two substituents.

In a first embodiment, ring A may suitably represent C₃₋₇ cycloalkyl, which group may be unsubstituted, or substituted by one or more substituents, typically by one or two substituents. As a general illustration of that embodiment, ring A may suitably represent cyclopropyl, cyclobutyl, cyclopentyl or cyclohexyl, any of which groups may be unsubstituted, or substituted by one or more substituents, typically by one or two substituents. As a particular illustration of that embodiment, ring A may suitably represent cyclopropyl or cyclobutyl, either of which groups may be unsubstituted, or substituted by one or more substituents, typically by one or two substituents. In a first aspect of that embodiment, ring A may suitably represent a cyclopropyl ring, which may be unsubstituted, or substituted by one or more substituents, typically by one or two substituents. In a second aspect of that embodiment, ring A may suitably represent a cyclobutyl ring, which may be unsubstituted, or substituted by one or more substituents, typically by one or two substituents. In a third aspect of that embodiment, ring A may suitably represent a cyclopentyl ring, which may be unsubstituted, or substituted by one or more substituents, typically by one or two substituents. In a fourth aspect of that embodiment, ring A may suitably represent a cyclohexyl ring, which may be unsubstituted, or substituted by one or more substituents, typically by one or two substituents.

In a second embodiment, ring A may suitably represent C₃₋₇ heterocycloalkyl, which group may be unsubstituted, or substituted by one or more substituents, typically by one or two substituents. As a representative illustration of that embodiment, ring A may suitably represent oxetanyl, tetrahydrofuranyl, pyrrolidinyl, tetrahydropyranyl or piperidinyl, any of which groups may be unsubstituted, or substituted by one or more substituents, typically by one or two substituents. As a general illustration of that embodiment, ring A may suitably represent oxetanyl, pyrrolidinyl, tetrahydropyranyl or piperidinyl, any of which groups may be unsubstituted, or substituted by one or more substituents, typically by one or two substituents. As a favoured illustration of that embodiment, ring A may suitably represent oxetanyl, tetrahydrofuranyl, tetrahydro-pyranyl or piperidinyl, any of which groups may be unsubstituted, or substituted by one or more substituents, typically by one or two substituents. As a particular illustration of that embodiment, ring A may suitably represent tetrahydropyranyl or piperidinyl, either of which groups may be unsubstituted, or substituted by one or more substituents, typically by one or two substituents. In a first aspect of that embodiment, ring A may suitably represent an oxetanyl ring, which may be unsubstituted, or substituted by one or more substituents, typically by one or two substituents. In a second aspect of that embodiment, ring A may suitably represent a tetrahydrofuranyl ring, which may be unsubstituted, or substituted by one or more substituents, typically by one or two substituents. In a third aspect of that embodiment, ring A may suitably represent a pyrrolidinyl ring, which may be unsubstituted, or substituted by one or more substituents, typically by one or two substituents. In a fourth aspect of that embodiment, ring A may suitably represent a tetrahydropyranyl ring, which may be unsubstituted, or substituted by one or more substituents, typically by one or two substituents. In a fifth aspect of that embodiment, ring A may suitably represent a piperidinyl ring, which may be unsubstituted, or substituted by one or more substituents, typically by one or two substituents. Generally, ring A may represent cyclopropyl, cyclobutyl, cyclopentyl, cyclohexyl, oxetanyl, tetrahydrofuranyl, pyrrolidinyl, tetrahydropyranyl or piperidinyl, any of which groups may be unsubstituted, or substituted by one or more substituents, typically by one or two substituents.

Typically, ring A may represent cyclopropyl, cyclobutyl, cyclopentyl, cyclohexyl, oxetanyl, pyrrolidinyl, tetrahydropyranyl or piperidinyl, any of which groups may be unsubstituted, or substituted by one or more substituents, typically by one or two substituents.

Favourably, ring A may represent cyclopropyl, cyclobutyl, oxetanyl, tetrahydro-furanyl, tetrahydropyranyl or piperidinyl, any of which groups may be unsubstituted, or substituted by one or more substituents, typically by one or two substituents.

Suitably, ring A may represent cyclopropyl, cyclobutyl, tetrahydropyranyl or piperidinyl, any of which groups may be unsubstituted, or substituted by one or more substituents, typically by one or two substituents.

Typical examples of optional substituents on ring A include one, two or three substituents independently selected from halogen, cyano, C₁₋₆ alkyl, fluoro(C₁₋₆)alkyl, difluoro(C₁₋₆)alkyl, trifluoro(C₁₋₆)alkyl, hydroxy, C₁₋₆ alkoxy, C₁₋₆ alkylthio, C₁₋₆ alkylsulfinyl, C₁₋₆ alkylsulfonyl, C₂₋₆ alkylcarbonyl, C₂₋₆ alkoxycarbonyl, amino, C₁₋₆ alkylamino and di(C₁₋₆)alkylamino.

Apposite examples of optional substituents on ring A include one, two or three substituents independently selected from halogen, C₁₋₆ alkyl, fluoro(C₁₋₆)alkyl, difluoro-(C₁₋₆)alkyl and C₂₋₆ alkoxycarbonyl.

Suitable examples of optional substituents on ring A include one, two or three substituents independently selected from halogen and C₂₋₆ alkoxycarbonyl.

Typical examples of particular substituents on ring A include one, two or three substituents independently selected from fluoro, chloro, bromo, cyano, methyl, fluoro-isobutyl, difluoropropyl, trifluoromethyl, trifluoroethyl, hydroxy, methoxy, methylthio, methylsulfinyl, methylsulfonyl, acetyl, methoxycarbonyl, ethoxycarbonyl, tert-butoxy-carbonyl, amino, methylamino and dimethylamino.

Apposite examples of particular substituents on ring A include one, two or three substituents independently selected from fluoro, methyl, fluoroisobutyl, difluoropropyl and tert-butoxycarbonyl.

Suitable examples of particular substituents on ring A include one, two or three substituents independently selected from fluoro and tert-butoxycarbonyl.

Typical examples of ring A include cyclopropyl, difluorocyclobutyl, cyclopentyl, difluorocyclohexyl, oxetanyl, methoxycarbonylpyrrolidinyl, tetrahydropyranyl, piperidinyl, methoxycarbonylpiperidinyl and tert-butoxycarbonylpiperidinyl. Additional examples include tetrahydrofuranyl, methylpiperidinyl, fluoroisobutylpiperidinyl and difluoropropylpiperidinyl.

Favoured examples of ring A include cyclopropyl, difluorocyclobutyl, oxetanyl, tetrahydrofuranyl, tetrahydropyranyl, piperidinyl, methylpiperidinyl, fluoroisobutyl-piperidinyl, difluoropropylpiperidinyl and tert-butoxycarbonylpiperidinyl.

Suitable examples of ring A include cyclopropyl, difluorocyclobutyl, tetrahydropyranyl, piperidinyl and tert-butoxycarbonylpiperidinyl.

In a first embodiment, R¹ represents hydrogen. In a second embodiment, R¹ represents fluoro. In a third embodiment, R¹ represents chloro. In a fourth embodiment, R¹ represents methyl. In a fifth embodiment, R¹ represents difluoromethyl. In a sixth embodiment, R¹ represents trifluoromethyl.

Typically, R¹ represents hydrogen, fluoro, chloro or methyl.

Generally, R¹ represents hydrogen or fluoro.

Suitably, R¹ represents hydrogen.

Typically, R⁶ represents —OR^{6a} or —NR^{6b}R^{6c}; or R⁶ represents C₁₋₆ alkyl, C₃₋₉ cycloalkyl, C₃₋₉ cycloalkyl(C₁₋₆)alkyl, aryl, aryl(C₁₋₆)alkyl, heteroaryl or heteroaryl-(C₁₋₆)alkyl, any of which groups may be optionally substituted by one or more substituents.

Appositely, R⁶ represents —OR^{6a} or —NR^{6b}R^{6c}; or R⁶ represents C₃₋₉ cycloalkyl or heteroaryl, either of which groups may be optionally substituted by one or more substituents.

Suitably, R⁶ represents —OR^{6a}; or R⁶ represents heteroaryl, which group may be optionally substituted by one or more substituents.

In a first embodiment, R⁶ represents optionally substituted C₁₋₆ alkyl. In a second embodiment, R⁶ represents optionally substituted C₃₋₉ cycloalkyl. In a third embodiment, R⁶ represents optionally substituted C₃₋₉ cycloalkyl(C₁₋₆)alkyl. In a fourth embodiment, R⁶ represents optionally substituted aryl. In a fifth embodiment, R⁶ represents optionally substituted aryl(C₁₋₆)alkyl. In a sixth embodiment, R⁶ represents optionally substituted C₃₋₇ heterocycloalkyl. In a seventh embodiment, R⁶ represents optionally substituted C₃₋₇ heterocycloalkyl(C₁₋₆)alkyl. In an eighth embodiment, R⁶ represents optionally substituted heteroaryl. In a ninth embodiment, R⁶ represents optionally substituted heteroaryl(C₁₋₆)alkyl. In a tenth embodiment, R⁶ represents —OR^{6a}. In an eleventh embodiment, R⁶ represents —NR^{6a}R^{6b}.

Typical examples of R⁶ include —OR^{6a} or —NR^{6a}R^{6b}; and methyl, ethyl, propyl, 2-methylpropyl, butyl, cyclopropyl, cyclobutyl, cyclohexyl, cyclohexylmethyl, phenyl, benzyl, phenylethyl, pyrazolyl, isoxazolyl, oxadiazolyl, triazolyl, pyridinyl, triazolyl-methyl, benzotriazolylmethyl or pyridinylmethyl, any of which groups may be optionally substituted by one or more substituents.

Representative examples of R⁶ include —OR^{6a} or —NR^{6a}R^{6b}; and cyclopropyl, pyrazolyl, oxadiazolyl or triazolyl, any of which groups may be optionally substituted by one or more substituents.

Illustrative examples of R⁶ include —OR^{6a}; and pyrazolyl, isoxazolyl, oxadiazolyl or triazolyl, any of which groups may be optionally substituted by one or more substituents.

Suitable examples of R⁶ include pyrazolyl, isoxazolyl, oxadiazolyl and triazolyl, any of which groups may be optionally substituted by one or more substituents.

Apposite examples of R⁶ include pyrazolyl, oxadiazolyl and triazolyl, any of which groups may be optionally substituted by one or more substituents.

Particular examples of R⁶ include oxadiazolyl, which group may be optionally substituted by one or more substituents.

Typical examples of optional substituents on R⁶ include one, two or three substituents independently selected from halogen, cyano, nitro, C₁₋₆ alkyl, difluoromethyl, trifluoromethyl, cyclopropyl, phenyl, fluorophenyl, hydroxy, hydroxy(C₁₋₆)alkyl, oxo, C₁₋₆ alkoxy, difluoromethoxy, trifluoromethoxy, C₁₋₆ alkylthio, C₁₋₆ alkylsulfinyl, C₁₋₆ alkylsulfonyl, amino, amino(C₁₋₆)alkyl, C₁₋₆ alkylamino, di(C₁₋₆)alkylamino, pyrrolidinyl, tetrahydropyranyl, morpholinyl, piperazinyl, C₂₋₆ alkylcarbonylamino, C₂₋₆ alkylcarbonyl-amino(C₁₋₆)alkyl, C₂₋₆ alkoxycarbonylamino, C₁₋₆ alkylsulfonylamino, formyl, C₂₋₆ alkyl-carbonyl, carboxy, C₂₋₆ alkoxycarbonyl, aminocarbonyl, C₁₋₆ alkylaminocarbonyl, di(C₁₋₆)alkylaminocarbonyl, aminosulfonyl, C₁₋₆ alkylaminosulfonyl, di(C₁₋₆)alkylamino-sulfonyl and di(C₁₋₆)alkylsulfoximinyl.

Suitable examples of optional substituents on R⁶ include one, two or three substituents independently selected from C₁₋₆ alkyl.

Typical examples of particular substituents on R⁶ include one, two or three substituents independently selected from fluoro, chloro, bromo, cyano, nitro, methyl, ethyl, isopropyl, tert-butyl, difluoromethyl, trifluoromethyl, cyclopropyl, phenyl, fluorophenyl, hydroxy, hydroxymethyl, oxo, methoxy, tert-butoxy, difluoromethoxy, trifluoromethoxy, methylthio, methylsulfinyl, methylsulfonyl, amino, aminomethyl, aminoethyl, methylamino, tert-butylamino, dimethylamino, pyrrolidinyl, tetrahydro-pyranyl, morpholinyl, piperazinyl, acetylamino, acetylaminoethyl, methoxycarbonyl-amino, methylsulfonylamino, formyl, acetyl, carboxy, methoxycarbonyl, ethoxycarbonyl, tert-butoxycarbonyl, aminocarbonyl, methylaminocarbonyl, dimethylaminocarbonyl, aminosulfonyl, methylaminosulfonyl, dimethylaminosulfonyl and dimethylsulfoximinyl.

Suitable examples of particular substituents on R⁶ include one, two or three substituents independently selected from methyl.

Illustrative examples of particular values of R⁶ include methyl, difluoromethyl, methylsulfonylmethyl, aminomethyl, methylaminomethyl, difluoroethyl, carboxyethyl, difluoropropyl, 2-methylpropyl, butyl, fluorocyclopropyl, cyanocyclopropyl, methyl-cyclopropyl, ethylcyclopropyl, dimethylcyclopropyl, difluoromethylcyclopropyl, trifluoromethylcyclopropyl, phenylcyclopropyl, fluorophenylcyclopropyl, hydroxy-cyclopropyl, aminocyclopropyl, cyclobutyl, trifluoromethylcyclobutyl, cyclohexyl, cyclohexylmethyl, phenyl, fluorophenyl, chlorophenyl, cyanophenyl, methylphenyl, hydroxyphenyl, methylsulfonylphenyl, dimethylsulfoximinylphenyl, benzyl, fluoro-benzyl, difluorobenzyl, chlorobenzyl, (chloro)(fluoro)benzyl, dichlorobenzyl, (chloro)-(difluoro)benzyl, bromobenzyl, cyanobenzyl, methylbenzyl, dimethylbenzyl, trifluoro-methylbenzyl, phenylbenzyl, hydroxybenzyl, hydroxymethylbenzyl, benzoyl, methoxy-benzyl, dimethoxybenzyl, trifluoromethoxybenzyl, methylsulfonylbenzyl, aminomethyl-benzyl, aminoethylbenzyl, dimethylaminobenzyl, pyrrolidinylbenzyl, (dimethyl)-(pyrrolidinyl)benzyl, morpholinylbenzyl, (dimethyl)(morpholinyl)benzyl, piperazinyl-benzyl, acetylaminoethylbenzyl, phenylethyl, chlorophenylethyl, methylpyrazolyl, ethylpyrazolyl, isopropylpyrazolyl, (methyl)(tetrahydropyranyl)pyrazolyl, methyl-isoxazolyl, ethylisoxazolyl, methyloxadiazolyl, ethyloxadiazolyl, cyclopropyloxadiazolyl, isopropyltriazolyl, pyridinyl, triazolylmethyl, benzotriazolylmethyl, pyridinylmethyl and aminopynridinylmethyl.

Favoured values of R⁶ include fluorocyclopropyl, difluoromethylcyclopropyl, trifluoromethylcyclopropyl, methylpyrazolyl, ethylpyrazolyl, isopropylpyrazolyl, methyl-isoxazolyl, ethylisoxazolyl, methyloxadiazolyl, ethyloxadiazolyl, cyclopropyloxadiazolyl and isopropyltriazolyl.

Typical values of R⁶ include fluorocyclopropyl, difluoromethylcyclopropyl, trifluoromethylcyclopropyl, isopropylpyrazolyl, methyloxadiazolyl, cyclopropyl-oxadiazolyl and isopropyltriazolyl.

Apposite values of R⁶ include isopropylpyrazolyl, methyloxadiazolyl, cyclopropyloxadiazolyl and isopropyltriazolyl.

A favoured value of R⁶ is methyloxadiazolyl.

Generally, R^{6a} represents C₁₋₆ alkyl; or R^{6a} represents C₃₋₉ cycloalkyl, which group may be optionally substituted by one or more substituents.

In a first embodiment, R^{6a} represents C₁₋₆ alkyl. In a second embodiment, R^{6a} represents optionally substituted C₃₋₉ cycloalkyl. In a third embodiment, R^{6a} represents optionally substituted C₃₋₇ heterocycloalkyl.

Appositely, R^{6a} represents C₁₋₆ alkyl; or R^{6a} represents cyclobutyl or oxetanyl, either of which groups may be optionally substituted by one or more substituents.

Typically, R^{6a} represents C₁₋₆ alkyl; or R^{6a} represents cyclobutyl, which group may be optionally substituted by one or more substituents.

Typical examples of optional substituents on R^{6a} include one, two or three substituents independently selected from halogen, cyano, nitro, C₁₋₆ alkyl, trifluoro-methyl, hydroxy, hydroxy(C₁₋₆)alkyl, oxo, C₁₋₆ alkoxy, difluoromethoxy, trifluoro-methoxy, C₁₋₆ alkylthio, C₁₋₆ alkylsulfinyl, C₁₋₆ alkylsulfonyl, amino, amino(C₁₋₆)alkyl, C₁₋₆ alkylamino, di(C₁₋₆)alkylamino, C₂₋₆ alkylcarbonylamino, C₂₋₆ alkoxycarbonylamino, C₁₋₆ alkylsulfonylamino, formyl, C₂₋₆ alkylcarbonyl, carboxy, C₂₋₆ alkoxycarbonyl, aminocarbonyl, C₁₋₆ alkylaminocarbonyl, di(C₁₋₆)alkylaminocarbonyl, aminosulfonyl, C₁₋₆ alkylaminosulfonyl and di(C₁₋₆)alkylaminosulfonyl.

Suitable examples of optional substituents on R^{6a} include one, two or three substituents independently selected from halogen.

Typical examples of specific substituents on R^{6a} include one, two or three substituents independently selected from fluoro, chloro, bromo, cyano, nitro, methyl, ethyl, isopropyl, tert-butyl, trifluoromethylhydroxy, hydroxymethyl, oxo, methoxy, tert-butoxy, difluoromethoxy, trifluoromethoxy, methylthio, methylsulfinyl, methylsulfonyl, amino, aminomethyl, aminoethyl, methylamino, tert-butylamino, dimethylamino, acetylamino, methoxycarbonylamino, methylsulfonylamino, formyl, acetyl, carboxy, methoxycarbonyl, ethoxycarbonyl, tert-butoxycarbonyl, aminocarbonyl, methylamino-carbonyl, dimethylaminocarbonyl, aminosulfonyl, methylaminosulfonyl and dimethyl-aminosulfonyl.

Suitable examples of specific substituents on R^{6a} include one, two or three substituents independently selected from fluoro.

Representative examples of specific values of R^{6a} include methyl, ethyl, n-propyl, isopropyl, n-butyl, tert-butyl, cyclobutyl, difluorocyclobutyl and oxetanyl.

Illustrative examples of specific values of R^{6a} include methyl, ethyl, n-propyl, isopropyl, n-butyl, tert-butyl, cyclobutyl and difluorocyclobutyl.

Typically, R^{6a} represents cyclobutyl.

Typically, R^{6b} represents hydrogen or methyl.

In a first embodiment, R^{6b} represents hydrogen. In a second embodiment, R^{6b} represents C₁₋₆ alkyl, especially methyl.

Typically, R^{6c} represents hydrogen or methyl.

In a first embodiment, R^{6c} represents hydrogen. In a second embodiment, R^{6c} represents C₁₋₆ alkyl, especially methyl.

Alternatively, the moiety —NR^{6b}R^{6c} may suitably represent azetidin-1-yl, pyrrolidin-1-yl, oxazolidin-3-yl, isoxazolidin-2-yl, thiazolidin-3-yl, isothiazolidin-2-yl, piperidin-1-yl, morpholin-4-yl, thiomorpholin-4-yl, piperazin-1-yl, homopiperidin-1-yl, homomorpholin-4-yl or homopiperazin-1-yl, any of which groups may be optionally substituted by one or more substituents.

Selected examples of suitable substituents on the heterocyclic moiety —NR^{6b}R^{6c} include C₁₋₆ alkyl, C₁₋₆ alkylsulfonyl, hydroxy, hydroxy(C₁₋₆)alkyl, amino(C₁₋₆)alkyl, cyano, oxo, C₂₋₆ alkylcarbonyl, carboxy, C₂₋₆ alkoxycarbonyl, amino, C₂₋₆ alkylcarbonyl-amino, C₂₋₆ alkylcarbonylamino(C₁₋₆)alkyl, C₂₋₆ alkoxycarbonylamino, C₁₋₆ alkylsulfonyl-amino and aminocarbonyl.

Selected examples of specific substituents on the heterocyclic moiety —NR^{6b}R^{6c} include methyl, methylsulfonyl, hydroxy, hydroxymethyl, aminomethyl, cyano, oxo, acetyl, carboxy, ethoxycarbonyl, amino, acetylamino, acetylaminomethyl, tert-butoxy-carbonylamino, methylsulfonylamino and aminocarbonyl.

A certain sub-class of compounds according to the invention is represented by the compounds of formula (IIA) and N-oxides thereof, and pharmaceutically acceptable salts thereof:

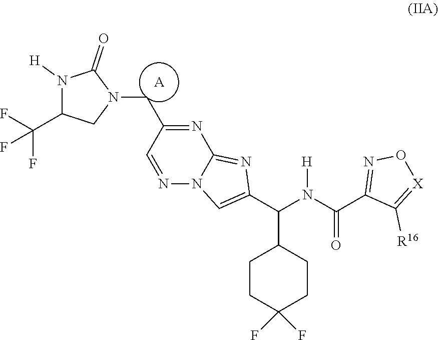

wherein
X represents CH or N;
R¹⁶ represents methyl, ethyl, isopropyl, difluoromethyl or cyclopropyl; and
A is as defined above.

In a first embodiment, X represents CH. In a second embodiment, X represents N.

In a first embodiment, R¹⁶ represents methyl. In a second embodiment, R¹⁶ represents ethyl. In a third embodiment, R¹⁶ represents isopropyl. In a fourth embodiment, R¹⁶ represents difluoromethyl. In a fifth embodiment, R¹⁶ represents cyclopropyl.

Suitably, R¹⁶ represents methyl, isopropyl or cyclopropyl, especially methyl.

Specific novel compounds in accordance with the present invention include each of the compounds whose preparation is described in the accompanying Examples, and pharmaceutically acceptable salts and solvates thereof.

The compounds in accordance with the present invention are beneficial in the treatment and/or prevention of various human ailments, including inflammatory and autoimmune disorders.

The compounds according to the present invention are useful in the treatment and/or prophylaxis of a pathological disorder that is mediated by a pro-inflammatory IL-17 cytokine or is associated with an increased level of a pro-inflammatory IL-17 cytokine. Generally, the pathological condition is selected from the group consisting of infections (viral, bacterial, fungal and parasitic), endotoxic shock associated with infection, arthritis, rheumatoid arthritis, psoriatic arthritis, systemic onset juvenile idiopathic arthritis (JIA), systemic lupus erythematosus (SLE), asthma, chronic obstructive airways disease (COAD), chronic obstructive pulmonary disease (COPD), acute lung injury, pelvic inflammatory disease, Alzheimer's Disease, Crohn's disease, inflammatory bowel disease, irritable bowel syndrome, ulcerative colitis, Castleman's disease, axial spondyloarthritis, ankylosing spondylitis and other spondyloarthropathies, dermatomyositis, myocarditis, uveitis, exophthalmos, autoimmune thyroiditis, Peyronie's Disease, coeliac disease, gall bladder disease, Pilonidal disease, peritonitis, psoriasis, atopic dermatitis, hidradenitis suppurativa, vasculitis, surgical adhesions, stroke, autoimmune diabetes, Type I Diabetes, lyme arthritis, meningoencephalitis, immune mediated inflammatory disorders of the central and peripheral nervous system such as multiple sclerosis and Guillain-Barr syndrome, other autoimmune disorders, pancreatitis, trauma (surgery), graft-versus-host disease, transplant rejection, fibrosing disorders including pulmonary fibrosis, liver fibrosis, renal fibrosis, scleroderma or systemic sclerosis, cancer (both solid tumours such as melanomas, hepatoblastomas, sarcomas, squamous cell carcinomas, transitional cell cancers, ovarian cancers and hematologic malignancies and in particular acute myelogenous leukaemia, chronic myelogenous leukemia, chronic lymphatic leukemia, gastric cancer and colon cancer), heart disease including ischaemic diseases such as myocardial infarction as well as atherosclerosis, intravascular coagulation, bone resorption, osteoporosis, periodontitis, hypochlorhydia and pain (particularly pain associated with inflammation).

WO 2009/089036 reveals that modulators of IL-17 activity may be administered to inhibit or reduce the severity of ocular inflammatory disorders, in particular ocular surface inflammatory disorders including Dry Eye Syndrome (DES). Consequently, the compounds in accordance with the present invention are useful in the treatment and/or prevention of an IL-17-mediated ocular inflammatory disorder, in particular an IL-17-mediated ocular surface inflammatory disorder including Dry Eye Syndrome. Ocular surface inflammatory disorders include Dry Eye Syndrome, penetrating keratoplasty, corneal transplantation, lamellar or partial thickness transplantation, selective endothelial transplantation, corneal neovascularization, keratoprosthesis surgery, corneal ocular surface inflammatory conditions, conjunctival scarring disorders, ocular autoimmune conditions, Pemphigoid syndrome, Stevens-Johnson syndrome, ocular allergy, severe allergic (atopic) eye disease, conjunctivitis and microbial keratitis. Particular categories of Dry Eye Syndrome include keratoconjunctivitis sicca (KCS), Sjögren syndrome, Sjögren syndrome-associated keratoconjunctivitis sicca, non-Sjögren syndrome-associated keratoconjunctivitis sicca, keratitis sicca, sicca syndrome, xerophthalmia, tear film disorder, decreased tear production, aqueous tear deficiency (ATD), meibomian gland dysfunction and evaporative loss.

Illustratively, the compounds of the present invention may be useful in the treatment and/or prophylaxis of a pathological disorder selected from the group consisting of arthritis, rheumatoid arthritis, psoriasis, psoriatic arthritis, systemic onset juvenile idiopathic arthritis (JIA), systemic lupus erythematosus (SLE), asthma, chronic obstructive airway disease, chronic obstructive pulmonary disease, atopic dermatitis, hidradenitis suppurativa, scleroderma, systemic sclerosis, lung fibrosis, inflammatory bowel diseases (including Crohn's disease and ulcerative colitis), axial spondyloarthritis, ankylosing spondylitis and other spondyloarthropathies, cancer and pain (particularly pain associated with inflammation).

Suitably, the compounds of the present invention are useful in the treatment and/or prophylaxis of psoriasis, psoriatic arthritis, hidradenitis suppurativa, axial spondylo-arthritis or ankylosing spondylitis.

The present invention also provides a pharmaceutical composition which comprises a compound in accordance with the invention as described above, or a pharmaceutically acceptable salt thereof, in association with one or more pharmaceutically acceptable carriers.

Pharmaceutical compositions according to the invention may take a form suitable for oral, buccal, parenteral, nasal, topical, ophthalmic or rectal administration, or a form suitable for administration by inhalation or insufflation.

For oral administration, the pharmaceutical compositions may take the form of, for example, tablets, lozenges or capsules prepared by conventional means with pharmaceutically acceptable excipients such as binding agents (e.g. pregelatinised maize starch, polyvinylpyrrolidone or hydroxypropyl methyl cellulose); fillers (e.g. lactose, microcrystalline cellulose or calcium hydrogenphosphate); lubricants (e.g. magnesium stearate, talc or silica); disintegrants (e.g. potato starch or sodium glycollate); or wetting agents (e.g. sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents, emulsifying agents, non-aqueous vehicles or preservatives. The preparations may also contain buffer salts, flavouring agents, colouring agents or sweetening agents, as appropriate.

Preparations for oral administration may be suitably formulated to give controlled release of the active compound.

For buccal administration, the compositions may take the form of tablets or lozenges formulated in conventional manner.

The compounds according to the present invention may be formulated for parenteral administration by injection, e.g. by bolus injection or infusion. Formulations for injection may be presented in unit dosage form, e.g. in glass ampoules or multi-dose containers, e.g. glass vials. The compositions for injection may take such forms as suspensions, solutions or emulsions in oily or aqueous vehicles, and may contain formulatory agents such as suspending, stabilising, preserving and/or dispersing agents. Alternatively, the active ingredient may be in powder form for constitution with a suitable vehicle, e.g. sterile pyrogen-free water, before use.

In addition to the formulations described above, the compounds according to the present invention may also be formulated as a depot preparation. Such long-acting formulations may be administered by implantation or by intramuscular injection.

For nasal administration or administration by inhalation, the compounds according to the present invention may be conveniently delivered in the form of an aerosol spray presentation for pressurised packs or a nebuliser, with the use of a suitable propellant, e.g. dichlorodifluoromethane, fluorotrichloromethane, dichlorotetrafluoroethane, carbon dioxide or other suitable gas or mixture of gases.

The compositions may, if desired, be presented in a pack or dispenser device which may contain one or more unit dosage forms containing the active ingredient. The pack or dispensing device may be accompanied by instructions for administration.

For topical administration the compounds according to the present invention may be conveniently formulated in a suitable ointment containing the active component suspended or dissolved in one or more pharmaceutically acceptable carriers. Particular carriers include, for example, mineral oil, liquid petroleum, propylene glycol, polyoxyethylene, polyoxypropylene, emulsifying wax and water. Alternatively, the compounds according to the present invention may be formulated in a suitable lotion containing the active component suspended or dissolved in one or more pharmaceutically acceptable carriers. Particular carriers include, for example, mineral oil, sorbitan monostearate, polysorbate 60, cetyl esters wax, cetearyl alcohol, benzyl alcohol, 2-octyldodecanol and water.

For ophthalmic administration the compounds according to the present invention may be conveniently formulated as micronized suspensions in isotonic, pH-adjusted sterile saline, either with or without a preservative such as a bactericidal or fungicidal agent, for example phenylmercuric nitrate, benzylalkonium chloride or chlorhexidine acetate. Alternatively, for ophthalmic administration the compounds according to the present invention may be formulated in an ointment such as petrolatum.

For rectal administration the compounds according to the present invention may be conveniently formulated as suppositories. These can be prepared by mixing the active component with a suitable non-irritating excipient which is solid at room temperature but liquid at rectal temperature and so will melt in the rectum to release the active component. Such materials include, for example, cocoa butter, beeswax and polyethylene glycols.

The quantity of a compound according to the present invention required for the prophylaxis or treatment of a particular condition will vary depending on the compound chosen and the condition of the patient to be treated. In general, however, daily dosages may range from around 10 ng/kg to 1000 mg/kg, typically from 100 ng/kg to 100 mg/kg, e.g. around 0.01 mg/kg to 40 mg/kg body weight, for oral or buccal administration, from around 10 ng/kg to 50 mg/kg body weight for parenteral administration, and from around 0.05 mg to around 1000 mg, e.g. from around 0.5 mg to around 1000 mg, for nasal administration or administration by inhalation or insufflation.

If desired, a compound in accordance with the present invention may be co-administered with another pharmaceutically active agent, e.g. an anti-inflammatory molecule.

The compounds of formula (I) above may be prepared by a process which comprises reacting a carboxylic acid of formula R⁶—CO₂H or a salt thereof, e.g. an alkali metal salt such as a lithium salt thereof, with a compound of formula (III):

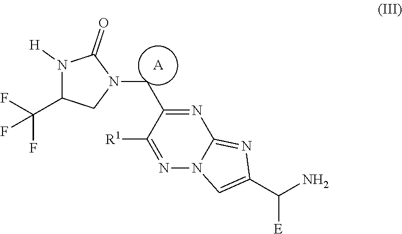

wherein E, A, R¹ and R⁶ are as defined above.

The reaction is conveniently accomplished in the presence of a coupling agent and a base. Suitable coupling agents include 1-[bis(dimethylamino)methylene]-1H-1,2,3-triazolo[4,5-b]pyridinium 3-oxid hexafluorophosphate (HATU); and 2,4,6-tripropyl-1,3,5,2,4,6-trioxatriphosphorinane-2,4,6-trioxide. Suitable bases include organic amines, e.g. a trialkylamine such as N,N-diisopropylethylamine; or pyridine. The reaction is conveniently performed at ambient or elevated temperature in a suitable solvent, e.g. a cyclic ether such as tetrahydrofuran; or a dipolar aprotic solvent such as N,N-dimethyl-formamide or N,N-dimethylacetamide; or a chlorinated solvent such as dichloromethane; or an organic ester solvent such as ethyl acetate.

Alternatively, the reaction may conveniently be accomplished in the presence of a coupling agent such as N-(3-dimethylaminopropyl)-N′-ethylcarbodiimide (EDC). The reaction is suitably performed at an appropriate temperature, e.g. a temperature in the region of 0° C., in a suitable solvent, e.g. an organic nitrile solvent such as acetonitrile.

Where R⁶ represents C₁₋₆ alkyl, e.g. methyl, the compounds of formula (I) above may be prepared by a process which comprises reacting a compound of formula R⁶—COCl, e.g. acetyl chloride, with a compound of formula (III) as defined above. The reaction is conveniently accomplished in the presence of a base. Suitable bases include organic amines, e.g. a trialkylamine such as N,N-diisopropylethylamine. The reaction is conveniently performed at ambient temperature in a suitable solvent, e.g. a cyclic ether such as tetrahydrofuran.

Where R⁶ represents —OR^{6a}, the compounds of formula (I) above may be prepared by a two-step process which comprises: (i) reacting a compound of formula R^{6a}—OH with N,N′-disuccinimidyl carbonate, ideally in the presence of a base, e.g. an organic amine such as triethylamine; and (ii) reacting the resulting material with a compound of formula (III) as defined above. Steps (i) and (ii) are conveniently performed at ambient temperature in a suitable solvent, e.g. a chlorinated solvent such as dichloromethane, or an organic nitrile solvent such as acetonitrile.

The intermediates of formula (III) above may be prepared by removal of the N-protecting group RP from a compound of formula (IV):

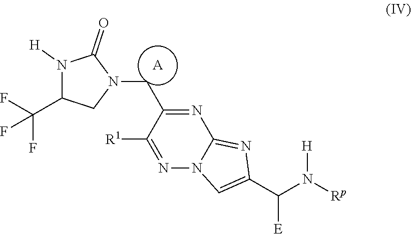

wherein E, A and R¹ are as defined above, and RP represents a N-protecting group.

The N-protecting group RP will suitably be tert-butoxycarbonyl (BOC), in which case the removal thereof may conveniently be effected by treatment with an acid, e.g. a mineral acid such as hydrochloric acid, or an organic acid such as trifluoroacetic acid.

Alternatively, the N-protecting group RP may be benzyloxycarbonyl, in which case the removal thereof may conveniently be effected by catalytic hydrogenation, typically by treatment with hydrogen gas or ammonium formate in the presence of a hydrogenation catalyst, e.g. palladium on charcoal, or palladium hydroxide on charcoal. In a variant method, where the N-protecting group RP is benzyloxycarbonyl, the removal thereof may be effected by treatment with boron tribromide; or by treatment with hydrogen bromide and acetic acid.

In an alternative procedure, the compounds of formula (I) above may be prepared by a two-step process which comprises:
(i) reacting 2-(trifluoromethyl)acrylic acid with a compound of formula (V):

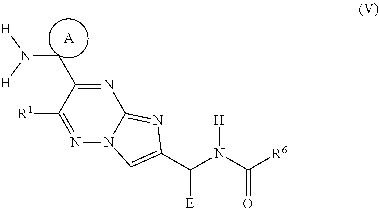

wherein E, A, R¹ and R⁶ are as defined above; and
(ii) treatment of the material thereby obtained with diphenyl phosphoryl azide.

Similarly, the intermediates of formula (IV) above may be prepared by a two-step process which comprises:
(i) reacting 2-(trifluoromethyl)acrylic acid with a compound of formula (VI):

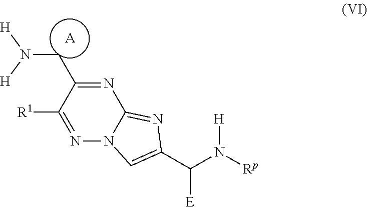

wherein E, A, R¹ and RP are as defined above; and
(ii) treatment of the material thereby obtained with diphenyl phosphoryl azide.

Step (i) is generally effected in the presence of a base. Suitable bases include alkali metal bicarbonates, e.g. sodium bicarbonate. The reaction is conveniently performed at ambient or elevated temperature in a suitable solvent, e.g. a C₁₋₄ alkanol such as methanol, or a cyclic ether such as 1,4-dioxane.

Step (ii) is generally effected in the presence of a base. Suitable bases include alkali metal tert-butoxides, e.g. sodium tert-butoxide. The reaction is conveniently performed at an elevated temperature in a suitable solvent, e.g. a cyclic ether such as 1,4-dioxane.

In another procedure, the compounds of formula (I) above may be prepared by a process which comprises reacting a compound of formula (VII) with a compound of formula (VIII):

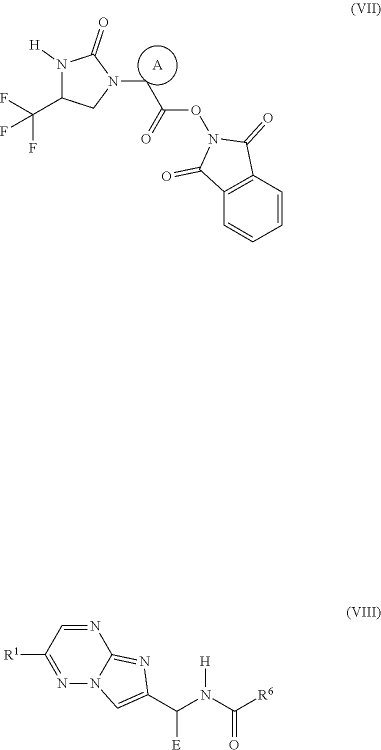

wherein E, A, R¹ and R⁶ are as defined above; in the presence of a transition metal catalyst and trifluoroacetic acid.

Suitable transition metal catalysts of use in the reaction include [4,4′-bis(1,1-dimethylethyl)-2,2′-bipyridine-N1,N1′]bis-{3,5-difluoro-2-[5-(trifluoromethyl)-2-pyridinyl-N]phenyl-C}iridium(III) hexafluorophosphate. The reaction will generally be performed by exposing the reactants to a bright light source. A suitable bright light source will typically comprise the ‘integrated photoreactor’ described in ACS Cent. Sci., 2017, 3, 647-653. The reaction will conveniently be carried out at ambient temperature in a suitable solvent, e.g. a dipolar aprotic solvent such as N,N-dimethylformamide; or an organic sulfoxide such as dimethyl sulfoxide.

The intermediates of formula (VII) above may be prepared by a two-step process which comprises:
(i) saponifying a compound of formula (IX):

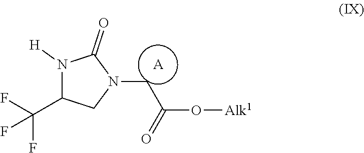

wherein A is as defined above, and Alk¹ represents C₁₋₄ alkyl, e.g. methyl, ethyl or tert-butyl; and
(ii) reaction of the carboxylic acid derivative thereby obtained with N-hydroxy-phthalimide.

Where Alk¹ represents methyl or ethyl, the saponification reaction in step (i) will generally be effected by treatment with a base. Suitable bases include inorganic hydroxides, e.g. an alkali metal hydroxide such as lithium hydroxide or sodium hydroxide. The reaction is conveniently performed at ambient or elevated temperature in water and a suitable organic solvent, e.g. a cyclic ether such as tetrahydrofuran, or a C₁₋₄ alkanol such as methanol.

Alternatively, where Alk¹ represents tert-butyl, the saponification reaction in step (i) may generally be effected by treatment with an acid, e.g. an organic acid such as trifluoroacetic acid. The reaction is conveniently performed at ambient temperature in a suitable organic solvent, e.g. a chlorinated solvent such as dichloromethane.

Step (ii) is generally effected in the presence of a coupling agent. Typical coupling agents include N-(3-dimethylaminopropyl)-N-ethylcarbodiimide hydrochloride (EDC·HCl); and N,N′-dicyclohexylcarbodiimide (DCC), suitably in the presence of 4-(dimethylamino)pyridine (DMAP). The reaction is conveniently performed at ambient temperature in a suitable solvent, e.g. a chlorinated solvent such as dichloromethane, or a cyclic ether such as tetrahydrofuran.

The intermediates of formula (IX) above may be prepared by a two-step process which comprises:
(i) reacting 2-(trifluoromethyl)acrylic acid with a compound of formula (X):

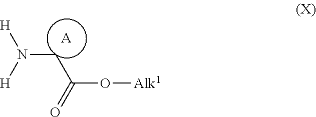

wherein A and Alk¹ are as defined above; under conditions analogous to those described above for the reaction between 2-(trifluoromethyl)acrylic acid and a compound of formula (V); and
(ii) treatment of the material thereby obtained with diphenyl phosphoryl azide; under conditions analogous to those described above.

The intermediates of formula (V) above may be prepared by a two-step process which comprises the following steps:
(i) reacting a compound of formula (VIII) as defined above with a compound of formula (XI):

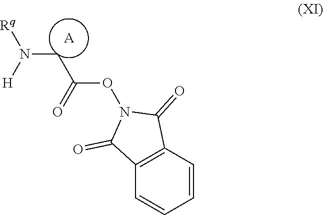

wherein A is as defined above, and R^{q} represents a N-protecting group; under conditions analogous to those described above for the reaction between compounds (VII) and (VIII); and
(ii) removal of the N-protecting group R^{q}.

Similarly, the intermediates of formula (VI) above may be prepared by a two-step process which comprises the following steps:
(i) reacting a compound of formula (XI) as defined above with a compound of formula (XII):

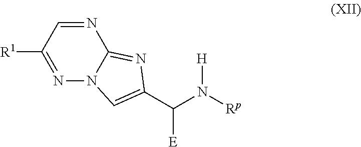

wherein E, R¹ and RP are as defined above; under conditions analogous to those described above for the reaction between compounds (VII) and (VIII); and
(ii) removal of the N-protecting group R^{q}.

The N-protecting group R^{q} will suitably be tert-butoxycarbonyl (BOC), in which case the removal thereof in step (ii) may conveniently be effected by treatment with an acid, e.g. a mineral acid such as hydrochloric acid, or an organic acid such as trifluoro-acetic acid.

The intermediates of formula (XI) above may be prepared by reacting a compound of formula (XIII):

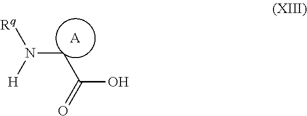

wherein A and R^{q} are as defined above; with N-hydroxyphthalimide; under conditions analogous to those described above for the reaction between N-hydroxyphthalimide and the carboxylic acid derivative obtained from the saponification of compound (IX).

The intermediates of formula (VIII) above may be prepared by a two-step process which comprises the following steps:
(i) removal of the N-protecting group RP from a compound of formula (XII) as defined above, under conditions analogous to those described above; and
(ii) reacting the compound thereby obtained with a carboxylic acid of formula R⁶—CO₂H or a salt thereof, e.g. an alkali metal salt such as a lithium salt thereof, under conditions analogous to those described above for the reaction between compound (III) and a carboxylic acid of formula R⁶—CO₂H or a salt thereof.

The intermediates of formula (XII) above may be prepared by reacting a compound of formula (XIV) with a compound of formula (XV):

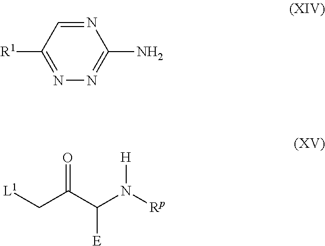

wherein E, R¹ and RP are as defined above, and L¹ represents a suitable leaving group.

The leaving group L¹ is typically a halogen atom, e.g. bromo.

The reaction is typically accomplished in the presence of a base. Suitably, the base may be an inorganic base, e.g. a bicarbonate salt such as sodium bicarbonate; or an organic base such as pyridine. The reaction is conveniently effected at an elevated temperature in a suitable solvent, e.g. a C₁₋₄ alkanol such as ethanol or isopropanol, or a cyclic ether such as 1,4-dioxane.

Where they are not commercially available, the starting materials of formula (X), (XIII), (XIV) and (XV) may be prepared by methods analogous to those described in the accompanying Examples, or by standard methods well known from the art.

It will be understood that any compound of formula (I) initially obtained from any of the above processes may, where appropriate, subsequently be elaborated into a further compound of formula (I) by techniques known from the art. By way of example, a compound comprising a N-BOC moiety (wherein BOC is an abbreviation for tert-butoxy-carbonyl) may be converted into the corresponding compound comprising a N—H moiety by treatment with an acid, e.g. a mineral acid such as hydrochloric acid, or an organic acid such as trifluoroacetic acid.

A compound comprising a N—H functionality may be alkylated, e.g. methylated, by treatment with a suitable alkyl halide, e.g. iodomethane, or by treatment with a suitable alkyl trifluoromethanesulfonate, typically in the presence of a base, e.g. an inorganic carbonate such as sodium carbonate or potassium carbonate.

A compound comprising a N—H functionality may be acylated, e.g. acetylated, by treatment with a suitable acyl halide, e.g. acetyl chloride, typically in the presence of a base, e.g. an organic base such as N,N-diisopropylethylamine or triethylamine. Similarly, a compound comprising a N—H functionality may be acylated, e.g. acetylated, by treatment with a suitable acyl anhydride, e.g. acetic anhydride, typically in the presence of a base, e.g. an organic base such as triethylamine.

Similarly, a compound comprising a N—H functionality may be converted into the corresponding compound comprising a N—S(O)₂Alk¹ functionality (wherein Alk¹ is as defined above) by treatment with the appropriate C₁₋₄ alkylsulfonyl chloride reagent, e.g. methylsulfonyl chloride, typically in the presence of a base, e.g. an organic base such as triethylamine.

Similarly, a compound comprising a N—H functionality may be converted into the corresponding compound comprising a carbamate or urea moiety respectively by treatment with the appropriate chloroformate or carbamoyl chloride reagent, typically in the presence of a base, e.g. an organic base such as triethylamine or N,N-diisopropylethyl-amine. Alternatively, a compound comprising a N—H functionality may be converted into the corresponding compound comprising a urea moiety by treatment with the appropriate amine-substituted (3-methylimidazol-3-ium-1-yl)methanone iodide derivative, typically in the presence of a base, e.g. an organic base such as triethylamine. Alternatively, a compound comprising a N—H functionality may be converted into the corresponding compound comprising a urea moiety N—C(O)N(H)Alk¹ (wherein Alk¹ is as defined above) by treatment with the appropriate isocyanate derivative Alk¹-N═C═O, typically in the presence of a base, e.g. an organic base such as triethylamine.

A compound comprising a N—H functionality may be converted into the corresponding compound comprising a N—C(H) functionality by treatment with the appropriate aldehyde or ketone in the presence of a reducing agent such as sodium triacetoxyborohydride.

A compound comprising a C₁₋₄ alkoxycarbonyl moiety —CO₂Alk¹ (wherein Alk¹ is as defined above) may be converted into the corresponding compound comprising a carboxylic acid (—CO₂H) moiety by treatment with a base, e.g. an alkali metal hydroxide salt such as lithium hydroxide. Alternatively, a compound comprising a tert-butoxy-carbonyl moiety may be converted into the corresponding compound comprising a carboxylic acid (—CO₂H) moiety by treatment with trifluoroacetic acid.

A compound comprising a carboxylic acid (—CO₂H) moiety may be converted into the corresponding compound comprising an amide moiety by treatment with the appropriate amine, under conditions analogous to those described above for the reaction between compound (III) and a carboxylic acid of formula R⁶—CO₂H.

A compound comprising a C₁₋₄ alkoxycarbonyl moiety —CO₂Alk¹ (wherein Alk¹ is as defined above) may be converted into the corresponding compound comprising a hydroxymethyl (—CH₂OH) moiety by treatment with a reducing agent such as lithium aluminium hydride.

A compound comprising a C₁₋₄ alkylcarbonyloxy moiety —OC(O)Alk¹ (wherein Alk¹ is as defined above), e.g. acetoxy, may be converted into the corresponding compound comprising a hydroxy (—OH) moiety by treatment with a base, e.g. an alkali metal hydroxide salt such as sodium hydroxide.

A compound comprising a halogen atom, e.g. bromo, may be converted into the corresponding compound comprising an optionally substituted aryl, heterocycloalkenyl or heteroaryl moiety by treatment with the appropriately substituted aryl, heterocycloalkenyl or heteroaryl boronic acid or a cyclic ester thereof formed with an organic diol, e.g. pinacol, 1,3-propanediol or neopentyl glycol. The reaction is typically effected in the presence of a transition metal catalyst, and a base. The transition metal catalyst may be [1,1′-bis(diphenylphosphino)ferrocene]dichloropalladium(II). In the alternative, the transition metal catalyst may be tris(dibenzylideneacetone)dipalladium(0), which may advantageously be employed in conjunction with 2-dicyclohexylphosphino-2′,4′,6′-triisopropylbiphenyl (XPhos). Suitably, the base may be an inorganic base such as sodium carbonate or potassium carbonate.

A compound comprising a halogen atom, e.g. bromo, may be converted into the corresponding compound comprising an optionally substituted aryl or heteroaryl moiety via a two-step procedure which comprises: (i) reaction with bis(pinacolato)diboron; and (ii) reaction of the compound thereby obtained with an appropriately substituted bromoaryl or bromoheteroaryl derivative. Step (i) is conveniently effected in the presence of a transition metal catalyst such as [1,1′-bis(diphenylphosphino)ferrocene]-dichloropalladium(II), and potassium acetate. Step (ii) is conveniently effected in the presence of a transition metal catalyst such as [1,1′-bis(diphenylphosphino)ferrocene]-dichloropalladium(II), and a base, e.g. an inorganic base such as sodium carbonate or potassium carbonate.

A compound comprising a cyano (—CN) moiety may be converted into the corresponding compound comprising a 1-aminoethyl moiety by a two-step process which comprises: (i) reaction with methylmagnesium chloride, ideally in the presence of titanium(IV) isopropoxide; and (ii) treatment of the resulting material with a reducing agent such as sodium borohydride. If an excess of methylmagnesium chloride is employed in step (i), the corresponding compound comprising a 1-amino-1-methylethyl moiety may be obtained.

A compound comprising the moiety —S— may be converted into the corresponding compound comprising the moiety —S(O)(NH)— by treatment with (diacetoxyiodo)benzene and ammonium carbamate.

A compound comprising a C═C double bond may be converted into the corresponding compound comprising a CH—CH single bond by treatment with gaseous hydrogen in the presence of a hydrogenation catalyst, e.g. palladium on charcoal.

A compound comprising an aromatic nitrogen atom may be converted into the corresponding compound comprising an N-oxide moiety by treatment with a suitable oxidising agent, e.g. 3-chloroperbenzoic acid.

Where a mixture of products is obtained from any of the processes described above for the preparation of compounds according to the invention, the desired product can be separated therefrom at an appropriate stage by conventional methods such as preparative HPLC; or column chromatography utilising, for example, silica and/or alumina in conjunction with an appropriate solvent system.

Where the above-described processes for the preparation of the compounds according to the invention give rise to mixtures of stereoisomers, these isomers may be separated by conventional techniques. In particular, where it is desired to obtain a particular enantiomer of a compound of formula (I) this may be produced from a corresponding mixture of enantiomers using any suitable conventional procedure for resolving enantiomers. Thus, for example, diastereomeric derivatives, e.g. salts, may be produced by reaction of a mixture of enantiomers of formula (I), e.g. a racemate, and an appropriate chiral compound, e.g. a chiral base. The diastereomers may then be separated by any convenient means, for example by crystallisation, and the desired enantiomer recovered, e.g. by treatment with an acid in the instance where the diastereomer is a salt. In another resolution process a racemate of formula (I) may be separated using chiral HPLC. Moreover, if desired, a particular enantiomer may be obtained by using an appropriate chiral intermediate in one of the processes described above. Alternatively, a particular enantiomer may be obtained by performing an enantiomer-specific enzymatic biotransformation, e.g. an ester hydrolysis using an esterase, and then purifying only the enantiomerically pure hydrolysed acid from the unreacted ester antipode. Chromatography, recrystallisation and other conventional separation procedures may also be used with intermediates or final products where it is desired to obtain a particular geometric isomer of the invention.

During any of the above synthetic sequences it may be necessary and/or desirable to protect sensitive or reactive groups on any of the molecules concerned. This may be achieved by means of conventional protecting groups, such as those described in Greene's Protective Groups in Organic Synthesis, ed. P. G. M. Wuts, John Wiley & Sons, 5^{th} edition, 2014. The protecting groups may be removed at any convenient subsequent stage utilising methods known from the art.

The compounds in accordance with this invention potently inhibit IL-17 induced IL-6 release from human dermal fibroblasts. Thus, when tested in the HDF cell line assay described below, compounds of the present invention exhibit a pIC₅₀ value of 5.0 or more, generally of 6.0 or more, usually of 7.0 or more, typically of 7.2 or more, suitably of 7.5 or more, ideally of 7.8 or more, and preferably of 8.0 or more (pIC₅₀ equals −log₁₀[IC₅₀], in which IC₅₀ is expressed as a molar concentration, so the skilled person will appreciate that a higher pIC₅₀ figure denotes a more active compound).

Inhibition of IL-17A Induced IL-6 Release from Dermal Fibroblast Cell Line

The purpose of this assay is to test the neutralising ability to IL-17 proteins, in a human primary cell system. Stimulation of normal human dermal fibroblasts (HDF) with IL-17 alone produces only a very weak signal but in combination with certain other cytokines, such as TNFα, a synergistic effect can be seen in the production of inflammatory cytokines, i.e. IL-6.

HDFs were stimulated with IL-17A (50 pM) in combination with TNF-α (25 pM). The resultant IL-6 response was then measured using a homogenous time-resolved FRET kit from Cisbio. The kit utilises two monoclonal antibodies, one labelled with Eu-Cryptate (Donor) and the second with d2 or XL665 (Acceptor). The intensity of the signal is proportional to the concentration of IL-6 present in the sample (Ratio is calculated by 665/620×104).

The ability of a compound to inhibit IL-17 induced IL-6 release from human dermal fibroblasts is measured in this assay.

HDF cells (Sigma #106-05n) were cultured in complete media (DMEM+10% FCS+2 mM L-glutamine) and maintained in a tissue culture flask using standard techniques. Cells were harvested from the tissue culture flask on the morning of the assay using TrypLE (Invitrogen #12605036). The TrypLE was neutralised using complete medium (45 mL) and the cells were centrifuged at 300×g for 3 minutes. The cells were re-suspended in complete media (5 mL) counted and adjusted to a concentration of 3.125×10⁴ cells/mL before being added to the 384 well assay plate (Corning #3701) at 40 μL per well. The cells were left for a minimum of three hours, at 37° C./5% CO₂, to adhere to the plate.

Compounds were serially diluted in DMSO before receiving an aqueous dilution into a 384 well dilution plate (Greiner #781281), where 5 pL from the titration plate was transferred to 45 μL of complete media and mixed to give a solution containing 10% DMSO.

Mixtures of TNFα and IL-17 cytokine were prepared in complete media to final concentrations of TNFα 25 pM/IL-17A 50 pM, then 30 μL of the solution was added to a 384 well reagent plate (Greiner #781281).

10 μL from the aqueous dilution plate was transferred to the reagent plate containing 30 μL of the diluted cytokines, to give a 2.5% DMSO solution. The compounds were incubated with the cytokine mixtures for 5 h at 37° C. After the incubation, 10 μL was transferred to the assay plate, to give a 0.5% DMSO solution, then incubated for 18-20 h at 37° C./5% CO₂.

From the Cisbio IL-6 FRET kit (Cisbio #62IL6PEB) europium cryptate and Alexa 665 were diluted in reconstitution buffer and mixed 1:1, as per kit insert. To a white low volume 384 well plate (Greiner #784075) were added FRET reagents (10 μL), then supernatant (10 μL) was transferred from the assay plate to Greiner reagent plate. The mixture was incubated at room temperature for 3 h with gentle shaking (<400 rpm) before being read on a Synergy Neo 2 plate reader (Excitation: 330 nm; Emission: 615/645 nm).

When tested in the HDF cell line assay as described above, the compounds of the accompanying Examples were found to exhibit the following PIC₅₀ values.

| Example | pIC₅₀ |
|---|---|
| 1 | 8.2 |
| 2 | 6.0 |
| 3 | 6.2 |
| 4 | 8.4 |
| 5 | 5.7 |
| 6 | 8.1 |
| 7 | 7.5 |
| 8 | 7.5 |
| 9 | 5.0 |
| 10 | 7.2 |
| 11 | 8.0 |
| 12 | 6.2 |
| 13 | 5.1 |
| 14 | 8.4 |
| 15 | 6.5 |
| 16 | 8.4 |
| 17 | 6.6 |
| 18 | 8.0 |
| 19 | 6.7 |

The following Examples illustrate the preparation of compounds according to the invention.

### EXAMPLES

### Abbreviations

DCM: dichloromethane
THF: tetrahydrofuran
MeOH: methanol
IPA: isopropanol
DMSO: dimethyl sulfoxide
DIPEA: N,N-diisopropylethylamine
DMF: N,N-dimethylformamide
EtOAc: ethyl acetate
TFA: trifluoroacetic acid
DPPA: diphenyl phosphoryl azide
DMAP: 4-(dimethylamino)pyridine
EDC·HCl: N-(3-dimethylaminopropyl)-N′-ethylcarbodiimide hydrochloride
HATU: 1-[bis(dimethylamino)methylene]-1H-1,2,3-triazolo[4,5-b]pyridinium 3-oxid hexafluorophosphate
{Ir[dF(CF₃)ppy]2(dtbpy)}PF₆: [4,4′-bis(1,1-dimethylethyl)-2,2′-bipyridine-N1,N1′]bis-{3,5-difluoro-2-[5-(trifluoromethyl)-2-pyridinyl-N]phenyl-C}iridium(III) hexafluoro-phosphate
h: hour
r.t.: room temperature
M: mass; molar
RT: retention time
HPLC: High Performance Liquid Chromatography
LCMS: Liquid Chromatography Mass Spectrometry
SFC: Supercritical Fluid Chromatography
ABPR: Automated back pressure regulator

### Analytical and Separation Methods

Method 1
X-Bridge C18 Waters 2.1 × 20 mm, 2.5 μm column

| Column Temperature: | 40° | C. |
|---|---|---|
| Mobile Phase A: | 10 mM ammonium formate in | |
| | water + 0.1% formic acid | |
| Mobile Phase B: | acetonitrile + 5% water + 0.1% formic acid | |
| Flow rate: | 1 | mL/minute |

Gradient program:

| Time | A % | B % |
|---|---|---|
| 0.00 | 95.00 | 5.00 |
| 1.50 | 5.00 | 95.00 |
| 2.25 | 5.00 | 95.00 |
| 2.50 | 95.00 | 5.00 |

Method 2
X-Bridge C18 Waters 2.1 × 20 mm, 2.5 μm column

| Column Temperature: | 40° | C. |
|---|---|---|
| Mobile Phase A: | 10 mM ammonium formate in | |
| | water + 0.1% formic acid | |
| Mobile Phase B: | acetonitrile + 5% water + 0.1% formic acid | |
| Flow rate: | 1 | mL/minute |

Gradient program:

| Time | A % | B % |
|---|---|---|
| 0.00 | 95.00 | 5.00 |
| 4.00 | 5.00 | 95.00 |
| 5.00 | 5.00 | 95.00 |
| 5.10 | 95.00 | 5.00 |

Method 3
Phenomenex Kinetex XB-C18 2.1 × 100 mm, 1.7 μm column

| Column Temperature: | 40° | C. |
|---|---|---|
| Mobile Phase A: | 0.1% formic acid in water | |
| Mobile Phase B: | 0.1% formic acid in acetonitrile | |
| Injection volume: | 1 | μL |
| Flow rate: | 0.6 | mL/minute |

Gradient program:

| Time | A % | B % |
|---|---|---|
| 0.00 | 95.00 | 5.00 |
| 5.30 | 0 | 100 |
| 5.80 | 0 | 100 |
| 5.82 | 95.00 | 5.00 |
| 7.00 | 95.00 | 5.00 |

Method 4
Waters UPLC ® BEH ™ C18, Part No.
186002352, 2.1 × 100 mm, 1.7 μm column

| Column Temperature: | 40° | C. |
|---|---|---|
| Mobile Phase A: | 2 mM ammonium bicarbonate | |
| | solution, buffered to pH 10 | |
| Mobile Phase B: | acetonitrile | |
| Flow rate: | 0.6 | mL/minute |

Gradient program:

| Time | A % | B % |
|---|---|---|
| 0.00 | 95.00 | 5.00 |
| 5.30 | 0 | 100 |
| 5.80 | 0 | 100 |
| 5.82 | 95.00 | 5.00 |
| 7.00 | 95.00 | 5.00 |

Method 5
Phenomenex Gemini NX-C18 2 × 20 mm, 3 μm column

| | |
|---|---|
| Mobile Phase A: | 10 mM ammonium formate in |
| | water + 0.1% ammonia solution |
| Mobile Phase B: | acetonitrile + 5% water + 0.1% ammonia solution |
| Flow rate: | 1 mL/minute |

Gradient program:

| Time | A % | B % |
|---|---|---|
| 0.00 | 95.00 | 5.00 |
| 1.50 | 5.00 | 95.00 |
| 2.25 | 5.00 | 95.00 |
| 2.50 | 95.00 | 5.00 |

Method 6
Waters Acquity UPLC BEH C18 2.1 × 50 mm, 1.7 μm column

| | |
|---|---|
| Mobile Phase A: | 10 mM ammonium formate in |
| | water + 0.1% ammonia solution |
| Mobile Phase B: | acetonitrile + 5% water + 0.1% ammonia solution |
| Flow rate: | 1.5 mL/minute |

Gradient program:

| Time | A % | B % |
|---|---|---|
| 0.00 | 95.00 | 5.00 |
| 0.10 | 95.00 | 5.00 |
| 3.50 | 5.00 | 95.00 |
| 4.00 | 5.00 | 95.00 |
| 4.05 | 95.00 | 5.00 |

Method 7

| | |
|---|---|
| Stationary phase: | Phenomenex Gemini NX-C18 |
| | 2 × 20 mm, 3 μm column |
| Mobile Phase A: | 10 mM ammonium formate in |
| | water + 0.1% ammonia solution |
| Mobile Phase B: | acetonitrile + 5% water + |
| | 0.1% ammonia solution |
| Flow rate: | 1 mL/minute |

Gradient program:

| Time | A % | B % |
|---|---|---|
| 0.00 | 95.00 | 5.00 |
| 4.00 | 5.00 | 95.00 |
| 5.00 | 5.00 | 95.00 |
| 5.10 | 95.00 | 5.00 |

Method 8
Waters SFC Prep 150 FractionLynx system, in
tandem with a Waters QDa mass spectrometer

| Stationary phase: | Chiralpak IC, 250 × 20.0 mm, 5 μm column | |
|---|---|---|
| Column temperature: | 40° | C. |
| Mobile Phase A: | CO₂ | |
| Mobile Phase B: | MeOH (+0.1% NH₄OH) | |
| Flow rate: | 100 | mL/minute |
| ABPR pressure: | 60 | bar |
| Run time: | 7.5 | minutes |

Gradient program:

| Time | A % | B % |
|---|---|---|
| 0.0 | 97.0 | 3.0 |
| 0.5 | 97.0 | 3.0 |
| 6.0 | 60.0 | 40.0 |
| 6.5 | 97.0 | 3.0 |

Method 9
Waters UPC² Acquity system, in tandem
with a Waters QDa mass spectrometer

| Stationary phase: | Chiralpak IC, 150 × 4.6 mm, 3 μm column | |
|---|---|---|
| Column temperature: | 35° | C. |
| Mobile Phase A: | CO₂ | |
| Mobile Phase B: | MeOH (+0.1% NH₄OH) | |
| Flow rate: | 3 | mL/minute |
| ABPR pressure: | 120 | bar |
| Run time: | 6.5 | minutes |

Gradient program:

| Time | A % | B % |
|---|---|---|
| 0.0 | 97.0 | 3.0 |
| 5.0 | 60.0 | 40.0 |
| 5.1 | 97.0 | 3.0 |

Method 10
Waters SFC Prep 150 FractionLynx system, in
tandem with a Waters QDa mass spectrometer

| Stationary phase: | Lux Cellulose-1, 250 × 21.2 mm, 5 μm column | |
|---|---|---|
| Column temperature: | 40° | C. |
| Mobile Phase A: | CO₂ | |
| Mobile Phase B: | MeOH (+ 0.1% NH₄OH) | |
| Flow rate: | 100 | mL/minute |
| ABPR pressure: | 120 | bar |
| Run time: | 15 | minutes |

Gradient program:

| Time | A % | B % |
|---|---|---|
| 0.0 | 90.0 | 10.0 |
| 15.0 | 90.0 | 10.0 |

Method 11
Waters UPC² Acquity system, in tandem
with a Waters QDa mass spectrometer

| Stationary phase: | Lux Cellulose-1, 150 × 4.6 mm, 3 μm column | |
|---|---|---|
| Column temperature: | 35° | C. |
| Mobile Phase A: | CO₂ | |
| Mobile Phase B: | MeOH (+0.1% NH₄OH) | |
| Flow rate: | 3 | mL/minute |
| ABPR pressure: | 120 | bar |
| Run time: | 6.5 | minutes |

Gradient program:

| Time | A % | B % |
|---|---|---|
| 0.0 | 97.0 | 3.0 |
| 5.0 | 60.0 | 40.0 |
| 5.1 | 97.0 | 3.0 |

### Intermediate 1

### Benzyl N—[(S)-(4,4-difluorocyclohexyl)(imidazo[1,2-b][1,2,4]triazin-6-yl)methyl]-carbamate

1,2,4-Triazin-3-amine (0.50 g, 5.20 mmol), benzyl N-[(1S)-3-bromo-1-(4,4-difluorocyclohexyl)-2-oxopropyl]carbamate (2.31 g, 5.72 mmol) and sodium hydrogen carbonate (1.31 g, 15.6 mmol) were heated in IPA (20 mL) at 80° C. with stirring for 18 h. The reaction mixture was filtered through Celite®, then concentrated under vacuum to near dryness. The material was dissolved in ethyl acetate (50 mL) and water (25 mL), then separated. The aqueous layer was extracted with additional ethyl acetate (25 mL). The combined organic extracts were washed with brine (10 mL), dried over sodium sulfate and concentrated under vacuum. The residue was purified by silica column chromatography, eluting with 0-10% MeOH in DCM, followed by reverse-phase C18 chromatography, eluting with 5-100% acetonitrile in water with 0.1% formic acid. The combined clean column fractions were saturated with NaHCO₃ (10 mL) and extracted with DCM (3×50 mL), to afford a first sample of material. Meanwhile, the impure fractions were further purified by silica column chromatography, eluting with 0-100% ethyl acetate in heptane, to afford a second sample of material. The first and second samples of material were combined, and the solvent was removed, to afford the title compound (570 mg, 27%) as a brown solid. LCMS (Method 1): [M+H]⁺ m/z 402.2, RT 1.84 minutes.

### Intermediate 2

### (1,3-Dioxoisoindolin-2-yl) 1-(tert-butoxycarbonylamino)-3,3-difluorocyclobutane-carboxylate

To a solution of 1-(tert-butoxycarbonylamino)-3,3-difluorocyclobutanecarboxylic acid (0.50 g, 1.99 mmol) in THF (10 mL) were added 2-hydroxy-1H-isoindole-1,3(2H)-dione (357 mg, 2.19 mmol), DMAP (24 mg, 0.199 mmol) and N,N′-dicyclohexyl-carbodiimide (493 mg, 2.39 mmol). The solution was stirred at r.t. for 18 h. The solids were filtered off, and the filtrate was concentrated. The crude residue was purified by silica column chromatography, eluting with 0-100% EtOAc in heptane, to give the title compound (609 mg, 77%) as a white solid. LCMS (Method 1): [2M+Na]⁺ m/z 815, RT 1.94 minutes.

### Intermediate 3

### tert-Butyl N-(1-{6-[(S)-benzyloxycarbonylamino(4,4-difluorocyclohexyl)methyl]-imidazo[1,2-b][1,2,4]triazin-3-yl}-3,3-difluorocyclobutyl)carbamate

A solution of Intermediate 1 (200 mg, 0.498 mmol), Intermediate 2 (296 mg, 0.747 mmol), {Ir[dF(CF₃)ppy]2(dtbpy)}PF₆ (11 mg, 9.96 μmol) and TFA (57 μL, 0.747 mmol) in anhydrous DMF (10 mL) was purged with nitrogen gas for 5 minutes. The reaction mixture was placed under 450 nm irradiation for 3 h, then diluted with EtOAc (30 mL) and washed with water (30 mL). The organic layer was dried over sodium sulfate and concentrated under vacuum. The residue was purified by silica column chromatography, eluting with 0-50% EtOAc in heptane, to afford the title compound (207 mg, 68%) as a pale yellow solid. LCMS (Method 1): [M+H]⁺ m/z 607.2, RT 2.05 minutes.

### Intermediate 4

### Benzyl N—{(S)-[3-(1-amino-3,3-difluorocyclobutyl)imidazo[1,2-b][1,2,4]triazin-6-yl](4,4-difluorocyclohexyl)methyl}carbamate

To a stirred solution of Intermediate 3 (207 mg, 0.341 mmol) in DCM (3 mL) was added TFA (0.42 mL, 5.46 mmol). The solution was stirred at r.t. for 3 h, then diluted with DCM and saturated aqueous NaHCO₃ solution (20 mL). The organic layer was separated. The aqueous layer was extracted with DCM (2×20 mL). The organic fractions were combined, then dried over sodium sulfate and concentrated under vacuum, to afford the title compound (170 mg, 98%) as a yellow solid. LCMS (Method 1): [M+H]⁺ m/z 507.2, RT 1.74 minutes.

### Intermediate 5

### 2-{[(1-{6-[(S)-Benzyloxycarbonylamino(4,4-difluorocyclohexyl)methyl]imidazo[1,2-b][1,2,4]triazin-3-yl}-3,3-difluorocyclobutyl)amino]methyl}-3,3,3-trifluoropropanoic acid

To Intermediate 4 (18 mg, 0.0355 mmol) in anhydrous MeOH (1 mL) in a sealed vial was added 2-(trifluoromethyl)prop-2-enoic acid (15 mg, 0.107 mmol) dropwise. The reaction mixture was stirred at r.t. for 16 h, then for 16 h at 40° C. Additional 2-(trifluoro-methyl)prop-2-enoic acid and NaHCO₃ (3 equivalents) were added, and the reaction mixture was stirred at r.t. for 48 h. Additional 2-(trifluoromethyl)prop-2-enoic acid and NaHCO₃ (3 equivalents) were again added. The reaction mixture was stirred at 50° C. for 16 h, then diluted with DCM (10 mL) and water (10 mL). The aqueous layer was acidified with 1M HCl to pH ˜6, then the organic layer was separated. The aqueous layer was extracted with DCM (2×10 mL). The organic fractions were combined, dried over sodium sulfate and concentrated under vacuum. The residue was purified by silica column chromatography, eluting with 0-10% MeOH in DCM, to afford the title compound (12 mg, 52%) as a pale yellow solid. LCMS (Method 1): [M+H]⁺ m/z 647.2, RT 2.36 minutes.

### Intermediate 6

### Benzyl N—[(S)-(4,4-difluorocyclohexyl)(3-{3,3-difluoro-1-[2-oxo-4-(trifluoromethyl)-imidazolidin-1-yl]cyclobutyl}imidazo[1,2-b][1,2,4]triazin-6-yl)methyl]carbamate

DPPA (0.15 mL, 0.711 mmol) was added to a solution of Intermediate 5 (115 mg, 0.178 mmol) and sodium 2-methylpropan-2-olate (51 mg, 0.534 mmol) in anhydrous 1,4-dioxane (3 mL) under nitrogen in a sealed vial. The reaction mixture was stirred at 80° C. for 5 h, then diluted with DCM (15 mL) and saturated aqueous NaHCO₃ solution (15 mL). The organic layer was separated. The aqueous layer was extracted with DCM (2×20 mL). The organic fractions were combined and dried over sodium sulfate, then concentrated under vacuum. The residue was purified by silica column chromatography, eluting with 0-100% EtOAc in heptane, to afford the title compound (57 mg, 50%) as a yellow solid. LCMS (Method 1): [M+H]⁺ m/z 644.2, RT 2.35 minutes.

### Intermediate 7

### 1-(1-{6-[(S)-Amino(4,4-difluorocyclohexyl)methyl]imidazo[1,2-b][1,2,4]triazin-3-yl}-3,3-difluorocyclobutyl)-4-(trifluoromethyl)imidazolidin-2-one

Hydrogen bromide (30% in acetic acid) (5.1M, 0.45 mL, 2.30 mmol) was added to a solution of Intermediate 6 (57 mg, 0.0886 mmol) in acetic acid (1.2 mL). The reaction mixture was stirred at r.t. for 3 h, then concentrated under vacuum. The crude material was dissolved in water (30 mL) and washed with EtOAc (3×10 mL). The aqueous layer was basified with solid NaHCO₃ and extracted with EtOAc (3×30 mL). The organic phase was dried over sodium sulfate, then filtered and concentrated in vacuo, to afford the title compound (44 mg, 85%) as a yellow gum. LCMS (Method 1): [M+H]⁺ m/z 510, RT 1.82 minutes.

### Intermediate 8

### (S)-(4,4-Difluorocyclohexyl)(imidazo[1,2-b][1,2,4]triazin-6-yl)methanamine

Intermediate 1 (3.00 g, 7.47 mmol) was dissolved in hydrogen bromide in acetic acid (35%) (25 mL, 0.154 mol). The reaction mixture was stirred at r.t. for 30 minutes, then additional hydrogen bromide in acetic acid (35%) (25 mL, 0.154 mol) and acetic acid (30 mL) were added. The reaction mixture was stirred for 1.5 h at r.t., then diethyl ether (100 mL) was added. The mixture was stirred for 30 minutes. The resulting precipitate was collected by vacuum filtration, then washed with diethyl ether, dissolved in water and washed with DCM. The aqueous portion was basified with saturated aqueous NaHCO₃ solution and extracted with DCM (3×100 mL). The combined organic extracts were washed with brine (50 mL) and dried over MgSO₄, then filtered and concentrated under reduced pressure, to afford the title compound (1.33 g, 60%) as a dark brown gum. LCMS (Method 1): [M+H]⁺ m/z 268.2, RT 1.27 minutes.

### Intermediate 9

### N—[(S)-(4,4-Difluorocyclohexyl)(imidazo[1,2-b][1,2,4]triazin-6-yl)methyl]-4-methyl-1,2,5-oxadiazole-3-carboxamide

A solution of Intermediate 8 (90%, 1.33 g, 4.48 mmol), 4-methyl-1,2,5-oxadiazole-3-carboxylic acid (631 mg, 4.93 mmol) and DIPEA (1.6 mL, 8.96 mmol) in anhydrous DMF (27 mL) was treated with HATU (2.04 g, 5.37 mmol) and stirred at r.t. for 1 h. The mixture was diluted with ethyl acetate (50 mL), washed with water (2×50 mL) and brine (10 mL), then dried over MgSO₄, filtered and concentrated under reduced pressure. The residue was purified by silica column chromatography, eluting with 50-80% EtOAc in heptane, to afford the title compound (1.39 g, 78%) as a light orange solid.

LCMS (Method 1): [M+H]⁺ m/z 378.2, RT 2.10 minutes.

### Intermediate 10

### (1,3-Dioxoisoindolin-2-yl) 1-(tert-butoxycarbonylamino)cyclopropanecarboxylate

EDC·HCl (1.00 g, 5.22 mmol) was added portionwise to a solution of 1-[(tert-butoxycarbonyl)amino]cyclopropanecarboxylic acid (1.00 g, 4.97 mmol) and 2-hydroxy-1H-isoindole-1,3(2H)-dione (0.89 g, 5.47 mmol) in DCM (20 mL) at 0° C. After 5 minutes, cooling was removed, and the reaction mixture was allowed to warm to r.t. overnight, then washed with water (50 mL). The aqueous phase was back-extracted with DCM (2×30 mL). The combined organic extracts were dried over sodium sulfate and concentrated to dryness. Purification by silica column chromatography, eluting with 10-100% EtOAc in heptanes, gave the title compound (0.54 g, 29%) as a white powder. LCMS (Method 1): [M+NH₄]⁺ m/z 364, RT 2.24 minutes.

### Intermediate 11

### tert-Butyl N-[1-(6-{(S)-(4,4-difluorocyclohexyl)[(4-methyl-1,2,5-oxadiazole-3-carbonyl)-amino]methyl}imidazo[1,2-b][1,2,4]triazin-3-yl)cyclopropyl]carbamate

A solution of Intermediate 9 (100 mg, 0.265 mmol), Intermediate 10 (87 mg, 0.252 mmol), {Ir[dF(CF₃)ppy]2(dtbpy)}PF₆ (5.7 mg, 5.05 μmol) and TFA (23 μL, 0.303 mmol) in anhydrous DMF (4.3 mL) was degassed with nitrogen. The reaction mixture was stirred at r.t. and irradiated at 450 nm for 6 h, then diluted with EtOAc (50 mL), and washed with saturated aqueous NaHCO₃ solution and water (3×20 mL). The organic phase was dried over sodium sulfate, filtered and concentrated to dryness under vacuum. The residue was purified by silica column chromatography, eluting with 10-100% EtOAc in heptanes, to afford the title compound (72 mg, 48%) as a yellow solid. LCMS (Method 2): [M+H]⁺ m/z 533, RT 4.08 minutes.

### Intermediate 12

### N—{(S)-[3-(1-Aminocyclopropyl)imidazo[1,2-b][1,2,4]triazin-6-yl](4,4-difluoro-cyclohexyl)methyl}-4-methyl-1,2,5-oxadiazole-3-carboxamide

TFA (0.50 mL, 6.53 mmol) was added to a stirred solution of Intermediate 11 (72 mg, 0.135 mmol) in DCM (2 mL). The solution was stirred at r.t. for 2 h, then concentrated under vacuum. The residue was purified by Isolute SCX-22 column, eluting with methanol, then 3.5M NH₃ in methanol, to afford the title compound (64 mg, 101%) as a yellow gum. LCMS (Method 1): [M+H]⁺ m/z 433, RT 1.80 minutes.

### Intermediate 13

### 2-({[1-(6-{(S)-(4,4-Difluorocyclohexyl)[(4-methyl-1,2,5-oxadiazole-3-carbonyl)amino]-methyl}imidazo[1,2-b][1,2,4]triazin-3-yl)cyclopropyl]amino}methyl)-3,3,3-trifluoro-propanoic acid

2-(Trifluoromethyl)prop-2-enoic acid (28 mg, 0.203 mmol) was added to a mixture of sodium hydrogen carbonate (34 mg, 0.406 mmol) and Intermediate 12 (58 mg, 0.135 mmol) in anhydrous 1,4-dioxane (1.35 mL) at r.t. The reaction mixture was stirred at r.t. for 16 h. The resulting material was utilised without further purification. LCMS (Method 1): [M+H]⁺ m/z 573, RT 2.19 minutes.

### Intermediate 14

### (1,3-Dioxoisoindolin-2-yl) 4-(tert-butoxycarbonylamino)tetrahydropyran-4-carboxylate

EDC·HCl (690 mg, 3.60 mmol) was added to a stirred suspension of 4-(tert-butoxycarbonylamino)tetrahydropyran-4-carboxylic acid (0.80 g, 3.26 mmol) and 2-hydroxy-1H-isoindole-1,3(2H)-dione (586 mg, 3.59 mmol) in anhydrous DCM (13 mL) at 0° C. under nitrogen. The mixture was stirred at 0° C. for 30 minutes, then at 20° C. for 1.5 h, then concentrated in vacuo. The residue was purified by flash column chromatography, eluting with 0-70% ethyl acetate in heptane, to afford the title compound (1.18 g, 90%) as a white powder. LCMS (Method 1): [M+NH₄]⁺ m/z 408.2, RT 2.20 minutes.

### Intermediate 15

### tert-Butyl N-[4-(6-{(S)-(4,4-difluorocyclohexyl)[(4-methyl-1,2,5-oxadiazole-3-carbonyl)-amino]methyl}imidazo[1,2-b][1,2,4]triazin-3-yl)tetrahydropyran-4-yl]carbamate

Intermediate 9 (160 mg, 0.40 mmol), Intermediate 14 (243 mg, 0.604 mmol) and {Ir[dF(CF₃)ppy]2(dtbpy)}PF₆ (9.1 mg, 8.1 μmol) were dissolved in anhydrous DMF (8 mL), and TFA (46.2 μL, 0.38 mmol) was added. The solution was degassed with nitrogen, sealed under nitrogen and irradiated with 450 nm LED light for 12 h. The reaction mixture was diluted with water (30 mL), quenched with saturated aqueous sodium hydrogen carbonate solution (20 mL) and extracted with ethyl acetate (3×30 mL). The combined organic extracts were washed with water (30 mL) and brine (30 mL), then dried over magnesium sulfate and concentrated in vacuo. The residue was purified by silica column chromatography, eluting with 0-100% ethyl acetate in heptane, to afford the title compound (51% purity) (119 mg) as an orange powder (containing a 38% impurity of Intermediate 9). LCMS (Method 2): [M+H]⁺ m/z 577.2, RT 3.92 minutes.

### Intermediate 16

### N—{(S)-[3-(4-Aminotetrahydropyran-4-yl)imidazo[1,2-b][1,2,4]triazin-6-yl](4,4-difluoro-cyclohexyl)methyl}-4-methyl-1,2,5-oxadiazole-3-carboxamide

TFA (0.40 mL, 5.22 mmol) was added to a stirred solution of Intermediate 15 (64%, 118 mg, 0.131 mmol) in DCM (1.5 mL). The solution was stirred at .rt. for 2 h, then concentrated under vacuum. The residue was purified by Isolute SCX-2 column, eluting with methanol, then 3.5M NH₃ in methanol, followed by silica column chromatography, eluting with 70-100% ethyl acetate in heptanes, followed by 10% methanol in ethyl acetate, to afford the title compound (92% NMR purity) (45 mg, 66%) as an orange solid. LCMS (Method 1): [M+H]⁺ m/z 477, RT 1.78 minutes.

### Intermediate 17

### 2-({[4-(6-{(S)-(4,4-Difluorocyclohexyl)[(4-methyl-1,2,5-oxadiazole-3-carbonyl)amino]-methyl}imidazo[1,2-b][1,2,4]triazin-3-yl)tetrahydropyran-4-yl]amino}methyl)-3,3,3-trifluoropropanoic acid

Intermediate 16 (45 mg, 0.0944 mmol), sodium hydrogen carbonate (20 mg, 0.236 mmol) and 2-(trifluoromethyl)prop-2-enoic acid (19 mg, 0.132 mmol) were stirred in 1,4-dioxane (1.5 mL) at r.t. overnight. The reaction mixture was retreated with 2-(trifluoro-methyl)prop-2-enoic acid (3.0 mg, 0.0214 mmol) and sodium hydrogen carbonate (3.0 mg, 0.0357 mmol), then stirred for another 2.5 h. The crude mixture (92% purity) was utilised without further purification. LCMS (Method 1): [M+H]⁺ m/z 617.2, RT 2.15 minutes.

### Intermediate 18

### O¹-tert-Butyl O⁴-methyl 4-[2-oxo-4-(trifluoromethyl)imidazolidin-1-yl]piperidine-1,4-dicarboxylate

Sodium bicarbonate (924 mg, 11.0 mmol) was added to a solution of 2-(trifluoro-methyl)acrylic acid (943 mg, 6.60 mmol) and 1-tert-butyl 4-methyl 4-aminopiperidine-1,4-dicarboxylate (1.50 g, 5.50 mmol) in 1,4-dioxane (55 mL). The reaction mixture was stirred at r.t. for 19 h, then 1,4-dioxane (55 mL) was added, followed by DPPA (2.93 mL, 13.2 mmol) and sodium tert-butoxide (793 mg, 8.25 mmol). Stirring was continued at 80° C. for another 4.5 h, then additional DPPA (2.93 mL, 13.2 mmol) and sodium tert-butoxide (793 mg, 8.25 mmol) were added. The reaction mixture was stirred at 80° C. for another 5.5 h, then concentrated in vacuo, diluted with DCM (100 mL) and washed with water (2×100 mL). The combined aqueous layers were extracted with DCM (2×50 mL). The organic layers were combined and washed with brine (50 mL), then passed through a phase separator and concentrated in vacuo. The crude residue was purified by flash column chromatography, eluting with a gradient of 0-100% EtOAc in hexanes, to give the title compound (1.62 g, 74%) as a pale yellow foam. δ_{H} (400 MHz, CDCl₃) 5.14 (s, 1H), 4.17-4.08 (m, 1H), 3.88-3.77 (m, 3H), 3.75 (s, 3H), 3.60 (dd, J 9.3, 3.7 Hz, 1H), 3.20-3.08 (m, 2H), 2.60-2.48 (m, 1H), 2.14-2.01 (m, 3H), 1.46 (s, 9H). LCMS (Method 5): [M−100+H]⁺ m/z 296.2, RT 1.20 minutes.

### Intermediate 19

### O¹-tert-Butyl O⁴-(1,3-dioxoisoindolin-2-yl) 4-[2-oxo-4-(trifluoromethyl)imidazolidin-1-yl]piperidine-1,4-dicarboxylate

Lithium hydroxide monohydrate (348 mg, 8.16 mmol) was added to a solution of Intermediate 18 (1.61 g, 4.08 mmol) in THF (16.3 mL) and water (4.1 mL). The reaction mixture was stirred at r.t. for 22 h, then concentrated in vacuo to remove organic solvents. The remaining aqueous solution was diluted with water (50 mL) and washed with EtOAc (50 mL). The organic layer was discarded, and the aqueous layer was adjusted to pH 3.5-4 by the addition of 2M aqueous HCl (3.5 mL). The acidified aqueous layer was extracted with EtOAc (3×50 mL). The organic layers were combined, passed through a phase separator and concentrated in vacuo. The resulting crude white foam (1.60 g) was dissolved in in DCM (19.3 mL) and N-hydroxyphthalimide (714 mg, 4.25 mmol) was added, followed by EDC·HCl (823 mg, 4.25 mmol). The reaction mixture was stirred at r.t. for 4 h, then additional N-hydroxyphthalimide (130 mg, 0.772 mmol) and EDC·HCl (150 mg, 0.773 mmol) were added. The reaction mixture was stirred at r.t. for a further 1.5 h, then concentrated in vacuo. The crude residue was purified by flash column chromatography, eluting with a gradient of 0-70% EtOAc in hexanes, to give the title compound (1.85 g, 86%) as a white foam. δ_{H} (400 MHz, CDCl₃) 7.90-7.85 (m, 2H), 7.82-7.77 (m, 2H), 5.31 (s, 1H), 4.26-4.16 (m, 1H), 3.92 (t, J 9.5 Hz, 1H), 3.84-3.72 (m, 2H), 3.69 (dd, J 9.4, 3.5 Hz, 1H), 3.48-3.34 (m, 2H), 2.79-2.65 (m, 1H), 2.43-2.27 (m, 2H), 2.25-2.15 (m, 1H), 1.47 (s, 9H). LCMS (Method 5): [M−100+H]⁺ m/z 427.2, RT 1.38 minutes.

### Intermediate 20

### (1,3-Dioxoisoindolin-2-yl) 3-(tert-butoxycarbonylamino)oxetane-3-carboxylate

To a solution of 3-(tert-butoxycarbonylamino)oxetane-3-carboxylic acid (400 mg, 1.84 mmol) and 2-hydroxy-1H-isoindole-1,3(2H)-dione (332 mg, 2.04 mmol) in DCM (8 mL) at 0° C. was added EDC·HCl (400 mg, 2.09 mmol). The reaction mixture was stirred for 5 minutes at 0° C., then at r.t. for 1 h. The mixture was re-treated with EDC·HCl (45 mg, 0.235 mmol) and stirred at r.t. overnight, then washed with water (15 mL). The aqueous layer was extracted with DCM (20 mL). The organic fractions were combined, dried over sodium sulphate and concentrated under vacuum. The residue was triturated in diethyl ether to afford the title compound (93% purity) (580 mg, 81%) as a white powder.

LCMS (Method 1): [M+NH₄]⁺ m/z 380.2, RT 2.15 minutes.

### Intermediate 21

### tert-Butyl N-[3-(6-{(S)-(4,4-difluorocyclohexyl)[(4-methyl-1,2,5-oxadiazole-3-carbonyl)-amino]methyl}imidazo[1,2-b][1,2,4]triazin-3-yl)oxetan-3-yl]carbamate

Intermediate 9 (280 mg, 0.742 mmol), Intermediate 20 (93% purity) (578 mg, 1.48 mmol) and {Ir[dF(CF₃)ppy]2(dtbpy)}PF₆ (17 mg, 14.8 μmol) were dissolved in DMSO (14 mL) and trifluoroacetic acid (85 mL, 1.11 mmol) was added. The reaction mixture was degassed with nitrogen, then sealed and irradiated with a 450 nm LED light for 20 h. The reaction mixture was diluted with EtOAc (20 mL) and washed with half-saturated aqueous sodium bicarbonate solution (2×10 mL). The aqueous layer was extracted with ethyl acetate (10 mL). The organic fractions were combined and washed with saturated brine (2×10 mL), then dried over sodium sulphate and concentrated under vacuum. The residue was purified by silica column chromatography, eluting with 35-55% ethyl acetate in heptane, to afford the title compound (64% purity) (564 mg) as an orange foam. LCMS (Method 1): [M+H]⁺ m/z 549.2, RT 2.26 minutes.

### Intermediate 22

### N—{(S)-[3-(3-Aminooxetan-3-yl)imidazo[1,2-b][1,2,4]triazin-6-yl](4,4-difluoro-cyclohexyl)methyl}-4-methyl-1,2,5-oxadiazole-3-carboxamide

To a solution of Intermediate 21 (64% purity) (464 mg, 0.541 mmol) in DCM (2 mL) was added trifluoroacetic acid (0.80 mL, 10.4 mmol). The reaction mixture was stirred for 3 h at r.t., then washed with half-saturated aqueous sodium bicarbonate solution (10 mL). The aqueous layer was extracted with DCM (10 mL), then the organic fractions were combined, dried over sodium sulphate and concentrated under vacuum. The residue was purified by reverse-phase C18 chromatography, eluting with 25-40% acetonitrile in water with 0.1% ammonia, to afford the title compound (96% purity) (229 mg, 91%) as a yellow solid. LCMS (Method 1): [M+H]⁺ m/z 449.2, RT 1.77 minutes.

### Intermediate 23

### 2-({[3-(6-{(S)-(4,4-Difluorocyclohexyl)[(4-methyl-1,2,5-oxadiazole-3-carbonyl)amino]-methyl}imidazo[1,2-b][1,2,4]triazin-3-yl)oxetan-3-yl]amino}methyl)-3,3,3-trifluoro-propanoic acid

Intermediate 22 (96% purity) (120 mg, 0.257 mmol), sodium hydrogen carbonate (43 mg, 0.514 mmol) and 2-(trifluoromethyl)prop-2-enoic acid (47 mg, 0.334 mmol) were stirred in 1,4-dioxane (2.5 mL) at r.t. for 2 h. The mixture was re-treated with 2-(trifluoromethyl)prop-2-enoic acid (47 mg, 0.334 mmol) and sodium hydrogen carbonate (43 mg, 0.514 mmol), then stirred overnight. The mixture was diluted with water (5 mL) and extracted with ethyl acetate (2×10 mL). The organic fractions were combined and washed with saturated brine (10 mL), then dried over sodium sulphate and concentrated under vacuum. The resulting yellow title compound was utilised without further purification. LCMS (Method 1): [M+H]⁺ m/z 589.2, RT 2.14 minutes.

### Intermediate 24

### tert-Butyl N-[3-(6-{(S)-(4,4-difluorocyclohexyl)[(4-methyl-1,2,5-oxadiazole-3-carbonyl)-amino]methyl}imidazo[1,2-b][1,2,4]triazin-3-yl)tetrahydrofuran-3-yl]carbamate

Nitrogen gas was bubbled through a solution of Intermediate 9 (200 mg, 0.53 mmol), (1,3-dioxoisoindolin-2-yl) 3-(tert-butoxycarbonylamino)tetrahydrofuran-3-carboxylate (299 mg, 0.80 mmol), {Ir[dF(CF₃)ppy]2(dtbpy)}PF₆ (12 mg, 0.01 mmol) and TFA (60 μL, 0.80 mmol) in DMF (5.3 mL) for 5 minutes. The reaction mixture was placed under 450 nm irradiation for 20 h, then diluted with EtOAc (25 mL) and washed with saturated aqueous NaHCO₃ solution (2×25 mL) and water (25 mL). The organic layer was passed through a phase separator, then concentrated in vacuo. The crude material was purified by silica column chromatography, eluting with a gradient of 0-100% EtOAc in isohexane, to give the title compound (121 mg, 40%) as a brown oil. LCMS (Method 7): [M+H]⁺ m/z 563.2, RT 2.11 minutes.

### Intermediate 25

### N—{(S)-[3-(3-Aminotetrahydrofuran-3-yl)imidazo[1,2-b][1,2,4]triazin-6-yl](4,4-difluoro-cyclohexyl)methyl}-4-methyl-1,2,5-oxadiazole-3-carboxamide

To a solution of Intermediate 24 (144 mg, 0.26 mmol) in DCM (1.0 mL) at r.t. was added TFA (0.26 mL). The reaction mixture was stirred for 2 h, then concentrated in vacuo. The crude residue was dissolved in DCM (20 mL) and saturated aqueous NaHCO₃ solution (20 mL). The aqueous layer was extracted with DCM (2×20 mL). The combined organic extracts were passed through a phase separator and concentrated in vacuo. The crude material was purified by silica column chromatography, eluting with a gradient of 0-100% EtOAc in isohexane, followed by 0-20% MeOH in EtOAc, to give the title compound (61 mg, 52%) as a brown amorphous solid. LCMS (Method 7): [M+H]⁺ m/z 463.2, RT 1.50 minutes.

### Examples 1 & 2

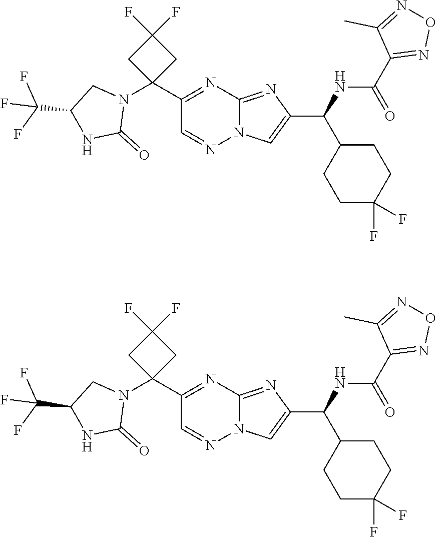 [SMILES: Cc1nonc1C(=O)N[C@H](c1cn2ncc(C3(N4C[C@@H](C(F)(F)F)NC4=O)CC(F)(F)C3)nc2n1)C1CCC(F)(F)CC1 ; Cc1nonc1C(=O)N[C@H](c1cn2ncc(C3(N4C[C@H](C(F)(F)F)NC4=O)CC(F)(F)C3)nc2n1)C1CCC(F)(F)CC1]

### N—[(S)-(4,4-Difluorocyclohexyl)(3-{3,3-difluoro-1-[(4R)-2-oxo-4-(trifluoromethyl)-imidazolidin-1-yl]cyclobutyl}imidazo[1,2-b][1,2,4]triazin-6-yl)methyl]-4-methyl-1,2,5-oxadiazole-3-carboxamide

HATU (56 mg, 0.148 mmol) was added to a mixture of 4-methyl-1,2,5-oxadiazole-3-carboxylic acid (19 mg, 0.148 mmol) and DIPEA (44 μL, 0.252 mmol) in anhydrous DMF (1 mL) at r.t. The reaction mixture was stirred at r.t. for 5 minutes, then a solution of Intermediate 7 (44 mg, 0.0872 mmol) in DMF (2 mL) was added. The reaction mixture was stirred at r.t. for 16 h, then diluted with EtOAc (50 mL) and washed with water (10 mL). The organic phase was dried over sodium sulfate, then filtered and concentrated to dryness. The residue was purified by low pH preparative HPLC to afford a mixture of the title compounds as an off-white solid. δ_{H} (400 MHz, CD₃OD) 8.70 (s, 1H), 8.19 (s, 1H), 5.28 (d, J 8.6 Hz, 1H), 4.44-4.33 (m, 1H), 3.97 (td, J 9.9, 1.5 Hz, 1H), 3.81 (dd, J 9.9, 3.6 Hz, 1H), 3.62-3.35 (m, 4H), 2.51 (s, 3H), 2.28 (d, J 8.9 Hz, 1H), 2.19-2.04 (m, 2H), 2.03-1.98 (m, 1H), 1.92-1.71 (m, 2H), 1.67 (d, J 14.7 Hz, 1H), 1.56-1.34 (m, 2H). Two NH signals not observed. LCMS (Method 3): [M+H]⁺ m/z 620, RT 3.44 minutes.

The foregoing material was subjected to SFC chromatography (Chiracel OD-H, 10×250 mm, 5 μm, 15:85 MeOH/CO₂, 15 mL/minute) to separate the mixture and provide the individual title compounds (Peak 1, 6.0 mg, 38%; and Peak 2, 5.4 mg, 33%) (absolute stereochemistry of carbon atom adjacent to CF₃ group unknown and arbitrarily assigned).

Peak 1 (Example 1): δ_{H} (500 MHz, CD₃OD) 8.70 (s, 1H), 8.20 (s, 1H), 5.29 (d, J 8.6 Hz, 1H), 4.45-4.32 (m, 1H), 3.97 (t, J 9.9 Hz, 1H), 3.81 (dd, J 10.0, 4.0 Hz, 1H), 3.63-3.38 (m, 3H), 2.52 (s, 3H), 2.28 (d, J 9.7 Hz, 1H), 2.15-1.95 (m, 2H), 1.95-1.60 (m, 3H), 1.60-1.24 (m, 3H). LCMS (Method 4): [M+H]⁺ m/z 620, RT 3.46 minutes. Chiral purity: 100% (SFC, Chiralcel OD-H, 4.6×250 mm, 5 μm, 15% methanol:85% CO₂, 4 mL/minute), RT 2.50 minutes.

Peak 2 (Example 2): δ_{H} (500 MHz, CD₃OD) 8.70 (s, 1H), 8.20 (s, 1H), 5.28 (d, J 8.6 Hz, 1H), 4.45-4.32 (m, 1H), 3.97 (t, J 9.9 Hz, 1H), 3.82 (dd, J 10.0, 4.0 Hz, 1H), 3.62-3.39 (m, 3H), 2.52 (s, 3H), 2.28 (d, J 9.2 Hz, 1H), 2.20-1.94 (m, 3H), 1.93-1.62 (m, 3H), 1.59-1.33 (m, 2H). LCMS (Method 4): [M+H]⁺ m/z 620, RT 3.47 minutes. Chiral purity: 100% (SFC, Chiralcel OD-H, 4.6×250 mm, 5 μm, 15% methanol:85% CO₂, 4 mL/minute), RT 4.89 minutes.

### Example 3

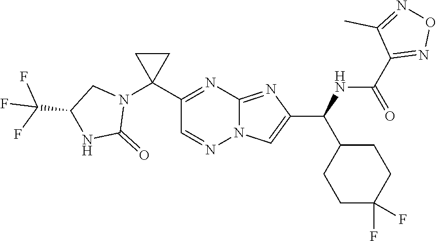 [SMILES: Cc1nonc1C(=O)N[C@H](c1cn2ncc(C3(N4C[C@@H](C(F)(F)F)NC4=O)CC3)nc2n1)C1CCC(F)(F)CC1]

### N—[(S)-(4,4-Difluorocyclohexyl)(3-{1-[(4S)-2-oxo-4-(trifluoromethyl)imidazolidin-1-yl]-cyclopropyl}imidazo[1,2-b][1,2,4]triazin-6-yl)methyl]-4-methyl-1,2,5-oxadiazole-3-carboxamide

DPPA (87 μL, 0.406 mmol) was added to a solution of Intermediate 13 (77 mg, 0.135 mmol) and sodium 2-methylpropan-2-olate (39 mg, 0.41 mmol) in anhydrous 1,4-dioxane (1.35 mL). The reaction mixture was stirred at 80° C. for 3 h, then additional DPPA (87 μL, 0.406 mmol) was added. The reaction mixture was stirred at 80° C. for a further 3.5 h, then diluted with DCM (30 mL) and washed with half-saturated aqueous NaHCO₃ solution (10 mL). The organic layer was separated. The aqueous layer was extracted with DCM (2×20 mL). The combined organic phases were dried over sodium sulfate and concentrated to dryness. The residue was purified by flash silica chromatography, eluting with 20-100% EtOAc in heptanes, then 0-20% MeOH in EtOAc, followed by achiral preparative HPLC, to afford a mixture of the title compound and its (4R) isomer (19 mg, 25%) as a pale yellow solid. δ_{H} (400 MHz, CD₃OD) 8.35 (s, 1H), 8.08 (s, 1H), 5.25 (d, J 8.5 Hz, 1H), 4.49-4.39 (m, 1H), 4.04 (t, J 10.1 Hz, 1H), 3.70 (dd, J 9.9, 3.9 Hz, 1H), 2.51 (s, 3H), 2.31-2.19 (m, 1H), 2.14-2.05 (m, 1H), 2.03-1.94 (m, 2H), 1.92-1.71 (m, 4H), 1.69-1.58 (m, 3H), 1.55-1.34 (m, 2H). Two NH signals not observed. LCMS (Method 3): [M+H]⁺ m/z 570, RT 3.15 minutes.

The foregoing material was subjected to SFC chromatography (Chiralpak IC, 10×250 mm, 5 μm, 25:75 MeOH/CO₂, 15 mL/minute) to separate the mixture and provide the title compound (Peak 1, 6.1 mg, 38%) (absolute stereochemistry of carbon atom adjacent to CF₃ group unknown and arbitrarily assigned) and its isomer (Peak 2).

Peak 1 (Example 3): δ_{H} (400 MHz, CD₃OD) 8.35 (s, 1H), 8.08 (s, 1H), 5.25 (d, J 8.5 Hz, 1H), 4.44 (ddd, J 10.3, 7.0, 4.1 Hz, 1H), 4.04 (t, J 10.1 Hz, 1H), 3.70 (dd, J 10.1, 4.1 Hz, 1H), 2.51 (s, 3H), 2.32-1.93 (m, 4H), 1.93-1.55 (m, 7H), 1.55-1.28 (m, 2H). LCMS (Method 4): [M+H]⁺ m/z 570, RT 3.21 minutes. Chiral purity: 100% (SFC, Chiralpak IC, 4.6×250 mm, 5 μm, 25% methanol:75% CO₂, 4 mL/minute), RT 2.75 minutes.

Peak 2: (SFC, Chiralpak IC, 4.6×250 mm, 5 μm 25% methanol:75% CO₂, 4 mL/minute) RT 6.77 minutes.

### Examples 4 & 5

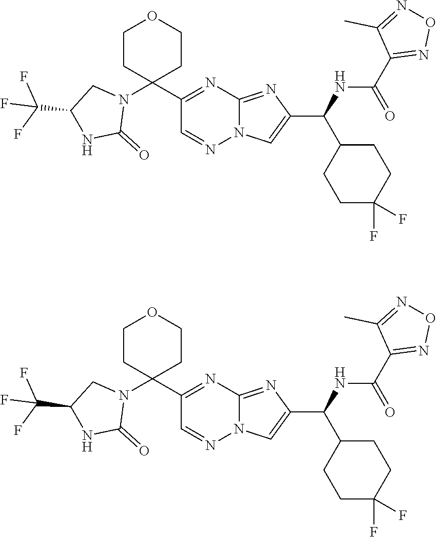 [SMILES: Cc1nonc1C(=O)N[C@H](c1cn2ncc(C3(N4C[C@@H](C(F)(F)F)NC4=O)CCOCC3)nc2n1)C1CCC(F)(F)CC1 ; Cc1nonc1C(=O)N[C@H](c1cn2ncc(C3(N4C[C@H](C(F)(F)F)NC4=O)CCOCC3)nc2n1)C1CCC(F)(F)CC1]

### N—[(S)-(4,4-Difluorocyclohexyl)(3-{4-[(4S)-2-oxo-4-(trifluoromethyl)imidazolidin-1-yl]-tetrahydropyran-4-yl}imidazo[1,2-b][1,2,4]triazin-6-yl)methyl]-4-methyl-1,2,5-oxadiazole-3-carboxamide

### N—[(S)-(4,4-Difluorocyclohexyl)(3-{4-[(4R)-2-oxo-4-(trifluoromethyl)imidazolidin-1-yl]-tetrahydropyran-4-yl}imidazo[1,2-b][1,2,4]triazin-6-yl)methyl]-4-methyl-1,2,5-oxadiazole-3-carboxamide

DPPA (37 μL, 0.173 mmol) was added to a solution of Intermediate 17 (92% purity) (58 mg, 0.0865 mmol) and sodium 2-methylpropan-2-olate (17 mg, 0.173 mmol) in 1,4-dioxane (2.5 mL) under nitrogen. The reaction mixture was stirred at 80° C. for 3 h, then re-treated with DPPA (37 μL, 0.173 mmol) and sodium 2-methylpropan-2-olate (17 mg, 0.173 mmol). The reaction mixture was stirred at 80° C. for another 2 h, then additional sodium 2-methylpropan-2-olate (3.3 mg, 0.0346 mmol) and DPPA (7.4 μL, 0.0346 mmol) were added. The mixture was stirred at 80° C. for 1 h, then cooled to r.t., diluted with ethyl acetate (10 mL) and washed with water (10 mL). The aqueous layer was extracted with ethyl acetate (10 mL). The organic fractions were combined, dried over sodium sulphate and concentrated under reduced pressure. The residue was purified by silica column chromatography, eluting with 35-50% acetonitrile in water (+0.1% formic acid), to give a mixture of the two isomeric title compounds (13 mg, 25%) as a white powder. δ_{H} (400 MHz, CD₃OD) 8.73 (s, 1H), 8.15 (s, 1H), 5.27 (d, J 8.5 Hz, 1H), 4.40-4.29 (m, 1H), 4.07 (t, J 9.9 Hz, 1H), 3.97-3.90 (m, 1H), 3.86-3.77 (m, 4H), 2.83-2.74 (m, 1H), 2.52 (s, 3H), 2.46-2.34 (m, 3H), 2.34-2.19 (m, 1H), 2.17-1.93 (m, 4H), 1.92-1.61 (m, 4H), 1.57-1.34 (m, 2H). LCMS (Method 3): [M+H]⁺ m/z 614.3, RT 3.12 minutes.

The foregoing material was subjected to SFC chromatography (Chiracel OD-H, 10×250 mm, 5 μm, 20:80 MeOH/CO₂, 15 mL/minute) to separate the mixture and provide the individual title compounds (Peak 1, 4.8 mg; and Peak 2, 4.2 mg) (absolute stereo-chemistry of carbon atom adjacent to CF₃ group unknown and arbitrarily assigned).

Peak 1 (Example 4): δ_{H} (400 MHz, CD₃OD) 8.73 (s, 1H), 8.15 (s, 1H), 5.27 (d, J 8.6 Hz, 1H), 4.40-4.29 (m, 1H), 4.07 (t, J 9.9 Hz, 1H), 3.97-3.88 (m, 1H), 3.88-3.76 (m, 4H), 2.81-2.72 (m, 1H), 2.52 (s, 3H), 2.45-2.34 (m, 3H), 2.33-2.19 (m, 1H), 2.17-1.98 (m, 3H), 1.93-1.71 (m, 2H), 1.71-1.63 (m, 1H), 1.56-1.34 (m, 2H). LCMS (Method 3): [M+H]⁺ m/z 614.3, RT 3.12 minutes. Chiral purity: 100% (SFC, Chiralcel OD-H, 4.6×250 mm, 5 μm, 25% methanol:75% CO₂, 4 mL/minute), RT 1.89 minutes.

Peak 2 (Example 5): δ_{H} (400 MHz, CD₃OD) 8.73 (s, 1H), 8.15 (s, 1H), 5.27 (d, J 8.6 Hz, 1H), 4.39-4.27 (m, 1H), 4.07 (t, J 9.9 Hz, 1H), 3.98-3.88 (m, 1H), 3.87-3.76 (m, 4H), 2.82-2.74 (m, 1H), 2.52 (s, 3H), 2.46-2.35 (m, 3H), 2.30-2.21 (m, 1H), 2.15-1.97 (m, 3H), 1.91-1.71 (m, 2H), 1.71-1.62 (m, 1H), 1.57-1.36 (m, 2H). LCMS (Method 3): [M+H]⁺ m/z 614.3, RT 3.12 minutes. Chiral purity: 100% (SFC, Chiralcel OD-H, 4.6×250 mm, 5 μm, 25% methanol:75% CO₂, 4 mL/minute), RT 2.77 minutes.

### Example 6

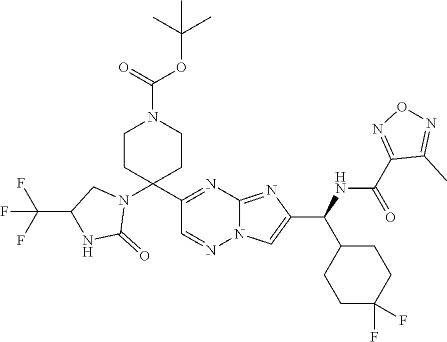 [SMILES: Cc1nonc1C(=O)N[C@H](c1cn2ncc(C3(N4CC(C(F)(F)F)NC4=O)CCN(C(=O)OC(C)(C)C)CC3)nc2n1)C1CCC(F)(F)CC1]

### tert-Butyl 4-(6-{(S)-(4,4-difluorocyclohexyl)[(4-methyl-1,2,5-oxadiazole-3-carbonyl)-amino]methyl}imidazo[1,2-b][1,2,4]triazin-3-yl)-4-[2-oxo-4-(trifluoromethyl)-imidazolidin-1-yl]piperidine-1-carboxylate

TFA (0.21 mL, 2.8 mmol) was added to a 40 mL screw-cap vial containing a solution of Intermediate 19 (1.09 g, 2.07 mmol), Intermediate 9 (520 mg, 1.38 mmol) and {Ir[dF(CF₃)ppy]2(dtbpy)}PF₆ (23.2 mg, 0.0207 mmol) in DMF (10.3 mL). The vial was sealed, and nitrogen was bubbled through the solution for 10 minutes, then the reaction mixture was irradiated using a Merck Penn PhD Photoreactor at 450 nm for 24 h. The reaction mixture was diluted with EtOAc (50 mL) and washed with water (3×50 mL).

The combined aqueous layers were extracted with EtOAc (2×25 mL). The organic layers were combined and washed with brine (50 mL), then passed through a phase separator and concentrated in vacuo. The crude residue was purified by flash column chromatography, eluting with a gradient of 0-100% EtOAc in hexanes, to give an orange oil (206 mg). A sample of that material (20.6 mg) was further purified by basic reverse phase preparative HPLC to afford, after lyophilisation, the title compound (7.6 mg, 37%) as a very pale yellow solid. δ_{H} (400 MHz, DMSO-d₆) 9.52 (dd, J 9.0, 3.0 Hz, 1H), 8.78 (d, J 1.5 Hz, 1H), 8.30 (d, J 1.0 Hz, 1H), 7.70 (s, 1H), 5.20 (t, J 8.6 Hz, 1H), 4.47-4.36 (m, 1H), 3.92 (td, J 9.9, 2.6 Hz, 1H), 3.74-3.57 (m, 3H), 3.29-3.17 (obs. m, 2H), 2.70-2.61 (m, 1H), 2.47 (s, 3H), 2.36-2.27 (m, 1H), 2.28-2.12 (m, 3H), 2.12-1.89 (m, 3H), 1.89-1.69 (m, 2H), 1.69-1.59 (m, 1H), 1.48-1.23 (m, 11H). LCMS (Method 6): [M+H]⁺ m/z 713.2, RT 2.17 minutes.

### Example 7

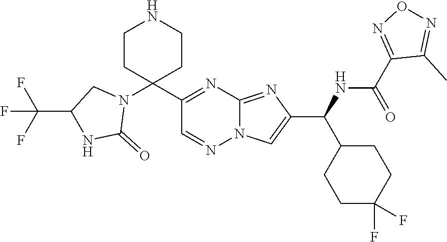 [SMILES: Cc1nonc1C(=O)N[C@H](c1cn2ncc(C3(N4CC(C(F)(F)F)NC4=O)CCNCC3)nc2n1)C1CCC(F)(F)CC1]

### N—[(S)-(4,4-Difluorocyclohexyl)(3-{4-[2-oxo-4-(trifluoromethyl)imidazolidin-1-yl]-piperidin-4-yl}imidazo[1,2-b][1,2,4]triazin-6-yl)methyl]-4-methyl-1,2,5-oxadiazole-3-carboxamide

Example 6 (16.6 mg, 0.0233 mmol) was dissolved in HCl in 1,4-dioxane (4 mol/L, 0.23 mL, 0.92 mmol). The reaction mixture was stirred at r.t. for 5 h, then concentrated in vacuo. The residue was purified by basic reverse-phase preparative HPLC to afford, after lyophilisation, the title compound (3.3 mg, 23%) as a very pale yellow solid. δ_{H} (400 MHz, DMSO-d₆) 9.51 (dd, J 9.1, 2.0 Hz, 1H), 8.74 (s, 1H), 8.29 (s, 1H), 7.61 (d, J 2.3 Hz, 1H), 5.20 (t, J 8.6 Hz, 1H), 4.48-4.36 (m, 1H), 3.98-3.90 (m, 1H), 3.67-3.61 (m, 1H), 2.91-2.70 (m, 4H), 2.62-2.53 (obs. m, 1H), 2.47 (s, 3H), 2.28-1.70 (m, 9H), 1.69-1.59 (m, 1H), 1.47-1.23 (m, 2H). The amine NH proton signal not observed. LCMS (Method 6): [M+H]⁺ m/z 613.2, RT 1.53 minutes.

### Examples 8 & 9

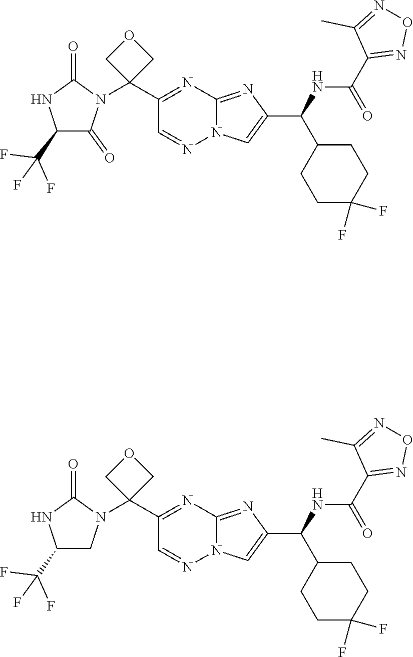 [SMILES: Cc1nonc1C(=O)N[C@H](c1cn2ncc(C3(N4C(=O)N[C@H](C(F)(F)F)C4=O)COC3)nc2n1)C1CCC(F)(F)CC1 ; Cc1nonc1C(=O)N[C@H](c1cn2ncc(C3(N4C[C@H](C(F)(F)F)NC4=O)COC3)nc2n1)C1CCC(F)(F)CC1]

### N—[(S)-(4,4-Difluorocyclohexyl)(3-{3-[(4S)-2-oxo-4-(trifluoromethyl)imidazolidin-1-yl]-oxetan-3-yl}imidazo[1,2-b][1,2,4]triazin-6-yl)methyl]-4-methyl-1,2,5-oxadiazole-3-carboxamide

### N—[(S)-(4,4-Difluorocyclohexyl)(3-{3-[(4R)-2-oxo-4-(trifluoromethyl)imidazolidin-1-yl]-oxetan-3-yl}imidazo[1,2-b][1,2,4]triazin-6-yl)methyl]-4-methyl-1,2,5-oxadiazole-3-carboxamide

DPPA (99 mL, 0.46 mmol) and 1,4-dioxane (2.5 mL) were added to Intermediate 23 (89% purity) (152 mg, 0.23 mmol). Sodium tert-butoxide (27 mg, 0.28 mmol) was added, and the reaction mixture was stirred at 80° C. for 1 h. The mixture was re-treated with DPPA (50 mL, 0.233 mmol) and sodium tert-butoxide (10 mg, 0.104 mmol), then stirred at 80° C. for another hour. The mixture was re-treated with more DPPA (50 mL, 0.233 mmol) and sodium tert-butoxide (10 mg, 0.104 mmol), then stirred at 80° C. for another hour. The mixture was diluted with half-saturated aqueous sodium bicarbonate solution (10 mL) and extracted with ethyl acetate (3×10 mL). The organic fractions were combined and washed with saturated brine (10 mL), then dried over sodium sulphate and concentrated under vacuum. The residue was purified by column chromatography, eluting with 50-70% ethyl acetate in heptane. The resulting yellow gum was further purified by reverse-phase C18 chromatography, eluting with 35-50% acetonitrile in water with 0.1% ammonia. The resulting yellow solid (29 mg) was subjected to SFC chromatography (Chiralpak AD-H, 10×250 mm, 5 μm column, 35:65 IPA:CO₂, 15 mL/minute) to afford the title compounds (absolute stereochemistry of carbon atom adjacent to CF₃ group unknown and arbitrarily assigned) as off-white solids.

Example 8 (first-eluting peak): δ_{H} (500 MHz, DMSO-d₆) 9.54 (d, J 8.9 Hz, 1H), 8.82 (s, 1H), 8.37 (s, 1H), 7.91 (d, J 2.4 Hz, 1H), 5.21 (t, J 8.6 Hz, 1H), 5.07-5.01 (m, 2H), 5.01-4.94 (m, 2H), 4.58-4.49 (m, 1H), 3.88 (t, J 9.8 Hz, 1H), 3.72 (dd, J 9.7, 4.0 Hz, 1H), 2.46 (s, 3H), 2.27-2.17 (m, 1H), 2.10-1.89 (m, 3H), 1.87-1.71 (m, 2H), 1.66-1.58 (m, 1H), 1.45-1.35 (m, 1H), 1.35-1.25 (m, 1H). LCMS (Method 4): [M+H]⁺ m/z 586.1, RT 3.04 minutes. Chiral purity: 100% (RT 1.96 minutes, SFC, 35% IPA, 65% CO₂, 4 mL/minute, Chiralpak AD-H, 4.6×250 mm, 5 μm column).

Example 9 (second-eluting peak): δ_{H} (500 MHz, DMSO-d₆) 9.53 (d, J 8.9 Hz, 1H), 8.82 (s, 1H), 8.36 (s, 1H), 7.91 (d, J 2.3 Hz, 1H), 5.21 (t, J 8.5 Hz, 1H), 5.07-4.97 (m, 3H), 4.97-4.93 (m, 1H), 4.58-4.50 (m, 1H), 3.89 (t, J 9.8 Hz, 1H), 3.72 (dd, J 9.7, 4.0 Hz, 1H), 2.46 (s, 3H), 2.28-2.18 (m, 1H), 2.10-1.89 (m, 3H), 1.87-1.70 (m, 2H), 1.66-1.58 (m, 1H), 1.45-1.35 (m, 1H), 1.35-1.25 (m, 1H). LCMS (Method 4): [M+H]⁺ m/z 586.1, RT 3.04 minutes. Chiral purity: 99% (RT 5.25 minutes, SFC, 35% IPA, 65% CO₂, 4 mL/minute, Chiralpak AD-H, 4.6×250 mm, 5 μm column).

### Examples 10 to 13

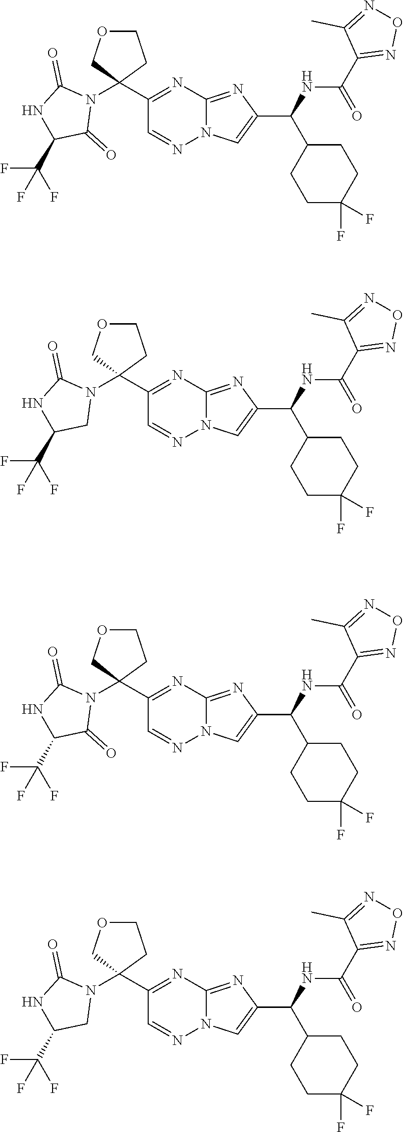 [SMILES: Cc1nonc1C(=O)N[C@H](c1cn2ncc([C@]3(N4C(=O)N[C@H](C(F)(F)F)C4=O)CCOC3)nc2n1)C1CCC(F)(F)CC1 ; Cc1nonc1C(=O)N[C@H](c1cn2ncc([C@@]3(N4C[C@@H](C(F)(F)F)NC4=O)CCOC3)nc2n1)C1CCC(F)(F)CC1 ; Cc1nonc1C(=O)N[C@H](c1cn2ncc([C@]3(N4C(=O)N[C@@H](C(F)(F)F)C4=O)CCOC3)nc2n1)C1CCC(F)(F)CC1 ; Cc1nonc1C(=O)N[C@H](c1cn2ncc([C@@]3(N4C[C@H](C(F)(F)F)NC4=O)CCOC3)nc2n1)C1CCC(F)(F)CC1]

### N—[(S)-(4,4-Difluorocyclohexyl)(3-{(3R)-3-[(4S)-2-oxo-4-(trifluoromethyl)imidazolidin-1-yl]tetrahydrofuran-3-yl}imidazo[1,2-b][1,2,4]triazin-6-yl)methyl]-4-methyl-1,2,5-oxadiazole-3-carboxamide

### N—[(S)-(4,4-Difluorocyclohexyl)(3-{(3S)-3-f[(4S)-2-oxo-4-(trifluoromethyl)imidazolidin-1-yl]tetrahydrofuran-3-yl}imidazo[1,2-b][1,2,4]triazin-6-yl)methyl]-4-methyl-1,2,5-oxadiazole-3-carboxamide

### N—[(S)-(4,4-Difluorocyclohexyl)(3-{(3R)-3-[(4R)-2-oxo-4-(trifluoromethyl)imidazolidin-1-yl]tetrahydrofuran-3-yl}imidazo[1,2-b][1,2,4]triazin-6-yl)methyl]-4-methyl-1,2,5-oxadiazole-3-carboxamide

### N—[(S)-(4,4-Difluorocyclohexyl)(3-{(3S)-3-[(4R)-2-oxo-4-(trifluoromethyl)imidazolidin-1-yl]tetrahydrofuran-3-yl}imidazo[1,2-b][1,2,4]triazin-6-yl)methyl]-4-methyl-1,2,5-oxadiazole-3-carboxamide

To a solution of Intermediate 25 (61 mg, 0.13 mmol) and 2-(trifluoromethyl)-acrylic acid (23 mg, 0.16 mmol) in 1,4-dioxane (1.3 mL) was added NaHCO₃ (22 mg, 0.26 mmol). The reaction mixture was stirred at r.t. for 24 h, then DPPA (70 μL, 0.32 mmol) and sodium tert-butoxide (19 mg, 0.20 mmol) were added. The reaction mixture was heated at 80° C. for 2 h, then cooled to r.t. Further portions of DPPA (70 μL, 0.32 mmol) and sodium tert-butoxide (19 mg, 0.20 mmol) were added, and the reaction mixture was stirred at 80° C. for a further 2 h. The reaction mixture was cooled to r.t., then diluted with water (20 mL) and extracted with DCM (3×20 mL). The combined organic extracts were passed through a phase separator and concentrated in vacuo. The crude material was purified by silica column chromatography, eluting with a gradient of 0-80% EtOAc in isohexane. The resulting off-white amorphous solid (52 mg) was subjected to chiral SFC (Lux Cellulose-4 250×21.5 mm, 5 μm column, eluting with 3-40% MeOH (+0.1% NH₄OH) at 100 mL/minute, 10 minute run time) to give the title compounds (Peak 1, 5.5 mg, 7%, RT 5.21 minutes; Peak 2, 6.1 mg, 8%, RT 5.45 minutes; Peak 3, 7.2 mg, 9%, RT 6.33 minutes; and Peak 4, 7.3 mg, 9%, RT 8.36 minutes) (absolute stereochemistry of carbon atom adjacent to CF₃ group, and quaternary carbon atom of tetrahydrofuranyl moiety, unknown and arbitrarily assigned) as colourless amorphous solids.

Example 10 (Peak 1): δ_{H} (400 MHz, DMSO-d₆) 9.51 (s, 1H), 8.64 (s, 1H), 8.31 (s, 1H), 7.82 (s, 1H), 5.23-5.15 (m, 1H), 4.54-4.44 (m, 1H), 4.26-4.18 (m, 2H), 4.00-3.93 (m, 3H), 3.80 (dd, J 10.0, 3.5 Hz, 1H), 2.81-2.74 (m, 1H), 2.55-2.50 (m, 1H), 2.47 (s, 3H), 2.27-2.17 (m, 1H), 2.11-1.90 (m, 3H), 1.88-1.70 (m, 2H), 1.66-1.59 (m, 1H), 1.46-1.23 (m, 2H). LCMS (Method 6): [M+H]⁺ m/z 600.2, RT 1.74 minutes. Chiral SFC (Lux Cellulose-4 150×4.6 mm, 3 μm column, flow rate 3 mL/minute, 120 bar, column temperature 35° C., eluting with a 3-40% MeOH (+0.1% NH₄OH) method, using a 6.5 minute run time on a Waters UPC2 Acquity system): RT 3.23 minutes (98%).

Example 11 (Peak 2): δ_{H} (400 MHz, DMSO-d₆) 9.52 (d, J 8.0 Hz, 1H), 8.70 (s, 1H), 8.31 (s, 1H), 7.77 (s, 1H), 5.22-5.18 (m, 1H), 4.54-4.47 (m, 1H), 4.22 (d, J 10 Hz, 1H), 4.10-4.05 (m, 2H), 4.01-3.90 (m, 2H), 3.74 (dd, J 10.0, 4.5 Hz, 1H), 2.84-2.76 (m, 1H), 2.65-2.58 (m, 1H), 2.47 (s, 3H), 2.26-2.17 (m, 1H), 2.11-1.89 (m, 3H), 1.89-1.70 (m, 2H), 1.68-1.59 (m, 1H), 1.45-1.24 (m, 2H). LCMS (Method 6): [M+H]⁺ m/z 600.2, RT 1.74 minutes. Chiral SFC (Lux Cellulose-4 150×4.6 mm, 3 μm column, flow rate 3 mL/minute, 120 bar, column temperature 35° C., eluting with a 3-40% MeOH (+0.1% NH₄OH) method, using a 6.5 minute run time on a Waters UPC2 Acquity system): RT 3.42 minutes (98%).

Example 12 (Peak 3): δ_{H} (400 MHz, DMSO-d₆) 9.50 (d, J 9.0 Hz, 1H), 8.64 (s, 1H), 8.31 (s, 1H), 7.82 (s, 1H), 5.22-5.18 (m, 1H), 4.54-4.45 (m, 1H), 4.25 (d, J 9.0 Hz, 1H), 4.19 (d, J 9.0 Hz, 1H), 4.00-3.94 (m, 3H), 3.81 (dd, J 10.0, 3.5 Hz, 1H), 2.81-2.74 (m, 1H), 2.56-2.49 (m, 1H), 2.47 (s, 3H), 2.27-2.17 (m, 1H), 2.11-1.90 (m, 3H), 1.88-1.70 (m, 2H), 1.67-1.59 (m, 1H), 1.46-1.22 (m, 2H). LCMS (Method 6): [M+H]⁺ m/z 600.2, RT 1.74 minutes. Chiral SFC (Lux Cellulose-4 150×4.6 mm, 3 μm column, flow rate 3 mL/minute, 120 bar, column temperature 35° C., eluting with a 3-40% MeOH (+0.1% NH₄OH) method, using a 6.5 minute run time on a Waters UPC2 Acquity system): RT 4.03 minutes (>99%).

Example 13 (Peak 4): δ_{H} (400 MHz, DMSO-d₆) 9.50 (d, J 9.0 Hz, 1H), 8.70 (s, 1H), 8.31 (s, 1H), 7.77 (s, 1H), 5.22-5.17 (m, 1H), 4.56-4.47 (m, 1H), 4.21 (d, J 10.5 Hz, 1H), 4.12-4.05 (m, 2H), 4.01-3.91 (m, 2H), 3.74 (dd, J 9.5, 4.5 Hz, 1H), 2.83-2.76 (m, 1H), 2.65-2.59 (m, 1H), 2.47 (s, 3H), 2.28-2.17 (m, 1H), 2.11-1.89 (m, 3H), 1.88-1.70 (m, 2H), 1.68-1.59 (m, 1H), 1.46-1.23 (m, 2H). LCMS (Method 6): [M+H]⁺ m/z 600.2, RT 1.73 minutes. Chiral SFC (Lux Cellulose-4 150×4.6 mm, 3 μm column, flow rate 3 mL/minute, 120 bar, column temperature 35° C., eluting with a 3-40% MeOH (+0.1% NH₄OH) method, using a 6.5 minute run time on a Waters UPC2 Acquity system): RT 5.44 minutes (>99%).

### Examples 14 & 15

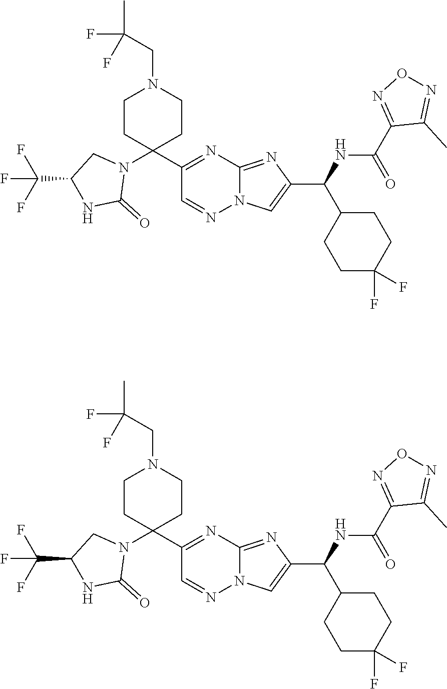 [SMILES: Cc1nonc1C(=O)N[C@H](c1cn2ncc(C3(N4C[C@@H](C(F)(F)F)NC4=O)CCN(CC(C)(F)F)CC3)nc2n1)C1CCC(F)(F)CC1 ; Cc1nonc1C(=O)N[C@H](c1cn2ncc(C3(N4C[C@H](C(F)(F)F)NC4=O)CCN(CC(C)(F)F)CC3)nc2n1)C1CCC(F)(F)CC1]

### N—[(S)-(4,4-Difluorocyclohexyl)(3-{1-(2,2-difluoropropyl)-4-[(4S)-2-oxo-4-(trifluoro-methyl)imidazolidin-1-yl]piperidin-4-yl}imidazo[1,2-b][1,2,4]triazin-6-yl)methyl]-4-methyl-1,2,5-oxadiazole-3-carboxamide

### N—[(S)-(4,4-Difluorocyclohexyl)(3-{1-(2,2-difluoropropyl)-4-[(4R)-2-oxo-4-(trifluoro-methyl)imidazolidin-1-yl]piperidin-4-yl}imidazo[1,2-b][1,2,4]triazin-6-yl)methyl]-4-methyl-1,2,5-oxadiazole-3-carboxamide

2,2-Difluoropropyl trifluoromethanesulfonate (18.7 mg, 0.082 mmol) was added to a solution of Example 7 (45.7 mg, 0.0746 mmol) and potassium carbonate (20.6 mg, 0.149 mmol) in acetonitrile (0.75 mL) under nitrogen. The reaction mixture was stirred at r.t. for 18.5 h. Additional potassium carbonate (10.3 mg, 0.0745 mmol) and 2,2-difluoro-propyl trifluoromethanesulfonate (8.5 mg, 0.037 mmol) were added, and the reaction mixture was stirred for a further 5.5 h. The reaction mixture was concentrated in vacuo, then re-dissolved in DCM (30 mL) and washed with saturated aqueous sodium bicarbonate solution (30 mL). The aqueous layer was extracted with DCM (2×15 mL), and the combined organic layers were passed through a phase separator and concentrated in vacuo. The crude residue was purified first by flash column chromatography, eluting with a gradient of 0-100% EtOAc in hexanes, then by chiral SFC (Method 8), to afford the title compounds (Peak 1, RT 4.44 minutes, 9.8 mg, 19%; and Peak 2, RT 5.57 minutes, 10.4 mg, 20%) (absolute stereochemistry of carbon atom adjacent to CF₃ group unknown and arbitrarily assigned) as pale yellow solids.

Example 14 (Peak 1): δ_{H} (400 MHz, DMSO-d₆) 9.53 (d, J 8.5 Hz, 1H), 8.78 (s, 1H), 8.30 (s, 1H), 7.65 (s, 1H), 5.20 (t, J 8.2 Hz, 1H), 4.46-4.35 (m, 1H), 3.92 (t, J 9.9 Hz, 1H), 3.63 (dd, J 10.1, 3.5 Hz, 1H), 2.85-2.66 (m, 5H), 2.62-2.49 (obs. m, 2H), 2.47 (s, 3H), 2.37-2.17 (m, 4H), 2.13-1.58 (m, 9H), 1.47-1.22 (m, 2H). LCMS (Method 6): [M+H]⁺ m/z 691.2, RT 2.08 minutes. Chiral SFC (Method 9): RT 3.60 minutes (100%).

Example 15 (Peak 2): δ_{H} (400 MHz, DMSO-d₆) 9.52 (d, J 7.4 Hz, 1H), 8.78 (s, 1H), 8.30 (s, 1H), 7.65 (s, 1H), 5.21 (t, J 7.5 Hz, 1H), 4.47-4.35 (m, 1H), 3.93 (t, J 9.9 Hz, 1H), 3.64 (dd, J 10.0, 3.5 Hz, 1H), 2.85-2.66 (m, 5H), 2.63-2.49 (obs. m, 2H), 2.47 (s, 3H), 2.36-2.17 (m, 4H), 2.12-1.58 (m, 9H), 1.47-1.22 (m, 2H). LCMS (Method 6): [M+H]⁺ m/z 691.2, RT 2.08 minutes. Chiral SFC (Method 9): RT 4.75 minutes (100%).

### Examples 16 & 17

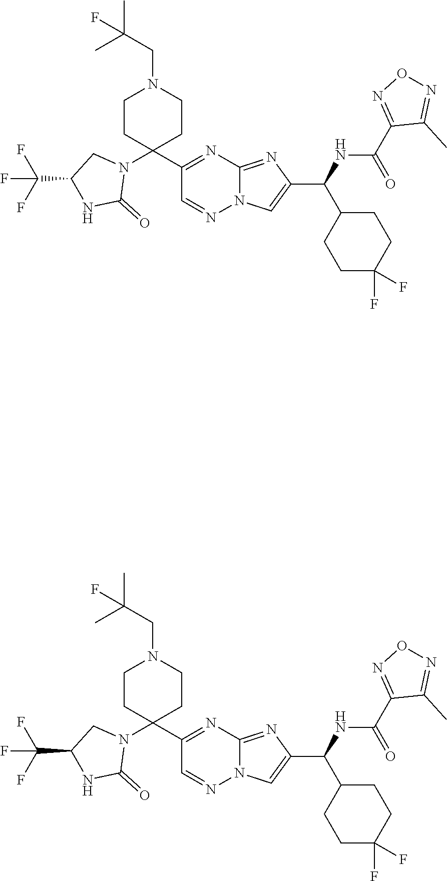 [SMILES: Cc1nonc1C(=O)N[C@H](c1cn2ncc(C3(N4C[C@@H](C(F)(F)F)NC4=O)CCN(CC(C)(C)F)CC3)nc2n1)C1CCC(F)(F)CC1 ; Cc1nonc1C(=O)N[C@H](c1cn2ncc(C3(N4C[C@H](C(F)(F)F)NC4=O)CCN(CC(C)(C)F)CC3)nc2n1)C1CCC(F)(F)CC1]

### N—[(S)-(4,4-Difluorocyclohexyl)(3-{1-(2-fluoro-2-methylpropyl)-4-[(4S)-2-oxo-4-(trifluoromethyl)imidazolidin-1-yl]piperidin-4-yl}imidazo[1,2-b][1,2,4]triazin-6-yl)-methyl]-4-methyl-1,2,5-oxadiazole-3-carboxamide

### N—[(S)-(4,4-Difluorocyclohexyl)(3-{1-(2-fluoro-2-methylpropyl)-4-[(4R)-2-oxo-4-(trifluoromethyl)imidazolidin-1-yl]piperidin-4-yl}imidazo[1,2-b][1,2,4]triazin-6-yl)-methyl]-4-methyl-1,2,5-oxadiazole-3-carboxamide

(2-Fluoro-2-methylpropyl) trifluoromethanesulfonate (19.4 mg, 0.0865 mmol) was added to a solution of Example 7 (48.2 mg, 0.0787 mmol) and potassium carbonate (21.7 mg, 0.157 mmol) in acetonitrile (0.79 mL) under nitrogen. The reaction mixture was stirred at r.t. for 18.5 h, then additional potassium carbonate (21.7 mg, 0.157 mmol) and (2-fluoro-2-methylpropyl) trifluoromethanesulfonate (19.4 mg, 0.0865 mmol) were added. The reaction mixture was stirred at r.t. for a further 24 h, then additional potassium carbonate (21.7 mg, 0.157 mmol) and (2-fluoro-2-methylpropyl) trifluoro-methanesulfonate (19.4 mg, 0.0865 mmol) were added. after 18.5 h and again after 42.5 h). The reaction mixture was stirred at r.t. for a further 5.5 h, then concentrated in vacuo. The residue was re-dissolved in DCM (30 mL) and washed with saturated aqueous sodium bicarbonate solution (30 mL). The aqueous layer was extracted with DCM (2×15 mL), and the combined organic layers were passed through a phase separator and concentrated in vacuo. The crude residue was purified first by flash column chromatography, eluting with a gradient of 0-100% EtOAc in hexanes, then by chiral SFC (Method 8), to afford the title compounds (Peak 1, RT 4.72 minutes, 7.8 mg, 14%; and Peak 2, RT 6.03 minutes, 6.3 mg, 12%) (absolute stereochemistry of carbon atom adjacent to CF₃ group unknown and arbitrarily assigned) as pale yellow solids.

Example 16 (Peak 1): δ_{H} (400 MHz, DMSO-d₆) 9.53 (d, J 8.2 Hz, 1H), 8.78 (s, 1H), 8.30 (s, 1H), 7.64 (s, 1H), 5.20 (t, J 7.9 Hz, 1H), 4.46-4.35 (m, 1H), 3.94 (t, J 9.9 Hz, 1H), 3.64 (dd, J 10.1, 3.4 Hz, 1H), 2.83-2.65 (m, 3H), 2.53-2.39 (obs. m, 7H), 2.35-2.16 (m, 4H), 2.13-1.70 (m, 5H), 1.68-1.59 (m, 1H), 1.48-1.22 (m, 8H). LCMS (Method 6): [M+H]⁺ m/z 687.2, RT 2.16 minutes. Chiral SFC (Method 9): RT 3.86 minutes (100%).

Example 17 (Peak 2): δ_{H} (400 MHz, DMSO-d₆) 9.52 (d, J 8.7 Hz, 1H), 8.78 (s, 1H), 8.30 (s, 1H), 7.63 (s, 1H), 5.21 (t, J 8.3 Hz, 1H), 4.46-4.35 (m, 1H), 3.94 (t, J 9.9 Hz, 1H), 3.65 (dd, J 10.1, 3.4 Hz, 1H), 2.82-2.65 (m, 3H), 2.53-2.39 (obs. m, 7H), 2.36-2.16 (m, 4H), 2.12-1.70 (m, 5H), 1.69-1.60 (m, 1H), 1.48-1.22 (m, 8H). LCMS (Method 6): [M+H]⁺ m/z 687.2, RT 2.16 minutes. Chiral SFC (Method 9): RT 5.20 minutes (100%).

### Examples 18 & 19

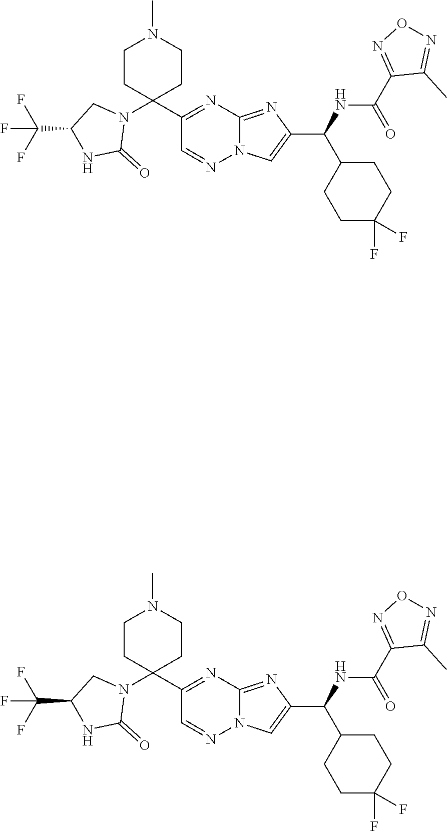 [SMILES: Cc1nonc1C(=O)N[C@H](c1cn2ncc(C3(N4C[C@@H](C(F)(F)F)NC4=O)CCN(C)CC3)nc2n1)C1CCC(F)(F)CC1 ; Cc1nonc1C(=O)N[C@H](c1cn2ncc(C3(N4C[C@H](C(F)(F)F)NC4=O)CCN(C)CC3)nc2n1)C1CCC(F)(F)CC1]

### N—[(S)-(4,4-Difluorocyclohexyl)(3-{1-methyl-4-[(4S)-2-oxo-4-(trifluoromethyl)-imidazolidin-1-yl]piperidin-4-yl}imidazo[1,2-b][1,2,4]triazin-6-yl)methyl]-4-methyl-1,2,5-oxadiazole-3-carboxamide

### N—[(S)-(4,4-Difluorocyclohexyl)(3-{1-methyl-4-[(4R)-2-oxo-4-(trifluoromethyl)-imidazolidin-1-yl]piperidin-4-yl}imidazo[1,2-b][1,2,4]triazin-6-yl)methyl]-4-methyl-1,2,5-oxadiazole-3-carboxamide

Formaldehyde (37 wt % in water with 15 wt % MeOH) (0.012 mL, 0.16 mmol) then acetic acid (0.009 mL, 0.2 mmol) were added to a solution of Example 7 (50.3 mg, 0.0821 mmol) in DCM (0.8 mL) and MeOH (0.16 mL). The reaction mixture was stirred at r.t. for 10 minutes, then sodium triacetoxyborohydride (34.8 mg, 0.164 mmol) was added. The reaction mixture was stirred at r.t. for a further 1.5 h, then diluted with DCM (20 mL) and saturated aqueous NaHCO₃ (20 mL). The layers were separated, and the aqueous layer was extracted with DCM (2×15 mL). The combined organic layers were passed through a phase separator and concentrated in vacuo. The crude residue was purified first by flash column chromatography, eluting with a gradient of 0-100% EtOAc in hexanes, followed by a gradient of 0-40% MeOH in EtOAc, then by chiral SFC (Method 10), to afford the title compounds (Peak 1, RT 6.99 minutes, 4.9 mg, 10%; and Peak 2, RT 9.13 minutes, 5.9 mg, 11%) (absolute stereochemistry of carbon atom adjacent to CF₃ group unknown and arbitrarily assigned) as very pale yellow solids.

Example 18 (Peak 1): δ_{H} (400 MHz, DMSO-d₆) 9.53 (d, J 8.8 Hz, 1H), 8.78 (s, 1H), 8.30 (s, 1H), 7.64 (s, 1H), 5.20 (t, J 8.5 Hz, 1H), 4.45-4.35 (m, 1H), 3.91 (t, J 9.9 Hz, 1H), 3.62 (dd, J 10.1, 3.4 Hz, 1H), 2.85-2.74 (m, 1H), 2.66-2.50 (obs. m, 2H), 2.47 (s, 3H), 2.36-2.15 (m, 9H), 2.12-1.90 (m, 3H), 1.89-1.70 (m, 2H), 1.68-1.59 (m, 1H), 1.47-1.20 (m, 2H). LCMS (Method 6): [M+H]⁺ m/z 627.2, RT 1.70 minutes. Chiral SFC (Method 11): RT 3.20 minutes (100%).

Example 19 (Peak 2): δ_{H} (400 MHz, DMSO-d₆) 9.53 (d, J 8.1 Hz, 1H), 8.78 (s, 1H), 8.30 (s, 1H), 7.64 (s, 1H), 5.20 (t, J 8.0 Hz, 1H), 4.46-4.35 (m, 1H), 3.92 (t, J 9.9 Hz, 1H), 3.63 (dd, J 10.0, 3.3 Hz, 1H), 2.85-2.74 (m, 1H), 2.66-2.50 (obs. m, 2H), 2.47 (s, 3H), 2.35-2.14 (m, 9H), 2.11-1.89 (m, 3H), 1.89-1.70 (m, 2H), 1.69-1.59 (m, 1H), 1.47-1.19 (m, 2H). LCMS (Method 6): [M+H]⁺ m/z 627.2, RT 1.70 minutes. Chiral SFC (Method 11): RT 3.45 minutes (98%).

1. A compound of formula (I) or an N-oxide thereof, or a pharmaceutically acceptable salt thereof: 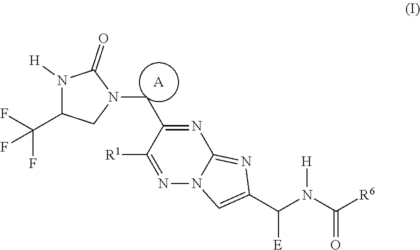
wherein
E represents a group of formula (Ea), (Eb), (Ec), (Ed) or (Ee): 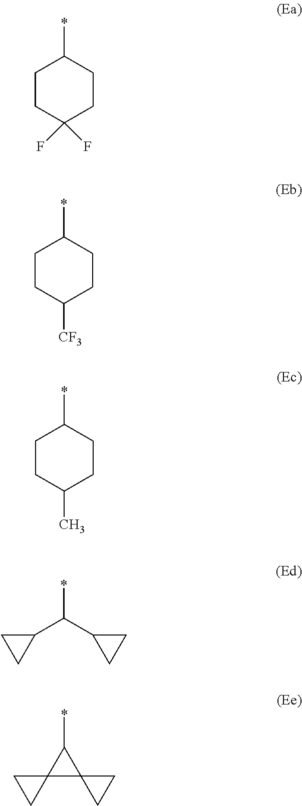
in which the asterisk (*) represents the point of attachment to the remainder of the molecule;
ring A represents C₃₋₇ cycloalkyl or C₃₋₇ heterocycloalkyl, either of which groups may be optionally substituted by one or more substituents;
R¹ represents hydrogen, fluoro, chloro, methyl, difluoromethyl or trifluoromethyl;
R⁶ represents —OR^{6a} or —NR^{6b}R^{6c}; or R⁶ represents C₁₋₆ alkyl, C₃₋₉ cycloalkyl, C₃₋₉ cycloalkyl(C₁₋₆)alkyl, aryl, aryl(C₁₋₆)alkyl, C₃₋₇ heterocycloalkyl, C₃₋₇ heterocycloalkyl-(C₁-6)alkyl, heteroaryl or heteroaryl(C₁₋₆)alkyl, any of which groups may be optionally substituted by one or more substituents;
R^{6a} represents C₁₋₆ alkyl; or R^{6a} represents C₃₋₉ cycloalkyl or C₃₋₇ heterocycloalkyl, either of which groups may be optionally substituted by one or more substituents;
R^{6b} represents hydrogen or C₁₋₆ alkyl; and
R^{6c} represents hydrogen or C₁₋₆ alkyl; or
R^{6b} and R^{6c}, when taken together with the nitrogen atom to which they are both attached, represent azetidin-1-yl, pyrrolidin-1-yl, oxazolidin-3-yl, isoxazolidin-2-yl, thiazolidin-3-yl, isothiazolidin-2-yl, piperidin-1-yl, morpholin-4-yl, thiomorpholin-4-yl, piperazin-1-yl, homopiperidin-1-yl, homomorpholin-4-yl or homopiperazin-1-yl, any of which groups may be optionally substituted by one or more substituents.

2. A compound as claimed in claim 1 wherein E represents a group of formula (Ea) or (Ed) as defined in claim 1.

3. A compound as claimed in claim 1 represented by formula (IA-1) or an N-oxide thereof, or a pharmaceutically acceptable salt thereof: 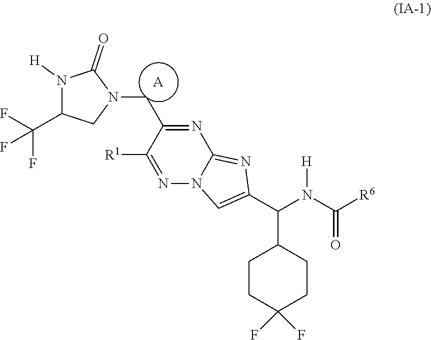
wherein
A, R¹ and R⁶ are as defined in claim 1.

4. A compound as claimed in claim 1 wherein R¹ represents hydrogen.

5. A compound as claimed in claim 1 wherein R⁶ represents heteroaryl, which group may be optionally substituted by one or more substituents.

6. A compound as claimed in claim 1 represented by formula (IIA) or an N-oxide thereof, or a pharmaceutically acceptable salt thereof: 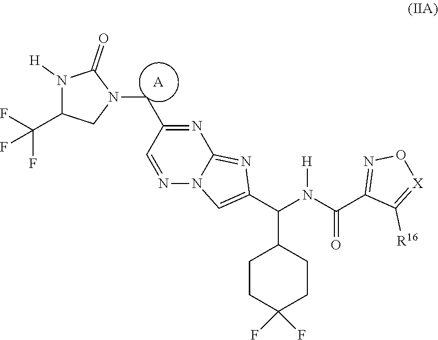
wherein
X represents CH or N;
R¹⁶ represents methyl, ethyl, isopropyl, difluoromethyl or cyclopropyl; and
A is as defined in claim 1.

7. A compound as claimed in claim 6 wherein R¹⁶ represents methyl.

8. A compound as claimed in claim 1 wherein ring A represents cyclopropyl, cyclobutyl, cyclopentyl, cyclohexyl, oxetanyl, tetrahydrofuranyl, pyrrolidinyl, tetrahydropyranyl or piperidinyl, any of which groups may be unsubstituted, or substituted by one, two or three substituents independently selected from halogen, cyano, C₁₋₆ alkyl, fluoro(C₁₋₆)alkyl, difluoro(C₁₋₆)alkyl, trifluoro(C₁₋₆)alkyl, hydroxy, C₁₋₆ alkoxy, C₁₋₆ alkylthio, C₁₋₆ alkylsulfinyl, C₁₋₆ alkylsulfonyl, C₂₋₆ alkylcarbonyl, C₂₋₆ alkoxycarbonyl, amino, C₁₋₆ alkylamino and di(C₁-6)alkylamino.

9. A compound as claimed in claim 8 wherein ring A represents cyclopropyl, cyclobutyl, oxetanyl, tetrahydrofuranyl, tetrahydropyranyl or piperidinyl, any of which groups may be unsubstituted, or substituted by one or two substituents independently selected from halogen, C₁₋₆ alkyl, fluoro(C₁₋₆)alkyl, difluoro(C₁₋₆)alkyl and C₂₋₆ alkoxy-carbonyl.

10. A compound as claimed in claim 1 as which is
N—[(S)-(4,4-Difluorocyclohexyl)(3-{3,3-difluoro-1-[(4S)-2-oxo-4-(trifluoromethyl)-imidazolidin-1-yl]cyclobutyl}imidazo[1,2-b][1,2,4]triazin-6-yl)methyl]-4-methyl-1,2,5-oxadiazole-3-carboxamide;
N—[(S)-(4,4-Difluorocyclohexyl)(3-{3,3-difluoro-1-[(4R)-2-oxo-4-(trifluoromethyl)-imidazolidin-1-yl]cyclobutyl}imidazo[1,2-b][1,2,4]triazin-6-yl)methyl]-4-methyl-1,2,5-oxadiazole-3-carboxamide;
N—[(S)-(4,4-Difluorocyclohexyl)(3-{1-[(4S)-2-oxo-4-(trifluoromethyl)imidazolidin-1-yl]-cyclopropyl}imidazo[1,2-b][1,2,4]triazin-6-yl)methyl]-4-methyl-1,2,5-oxadiazole-3-carboxamide;
N—[(S)-(4,4-Difluorocyclohexyl)(3-{4-[(4S)-2-oxo-4-(trifluoromethyl)imidazolidin-1-yl]-tetrahydropyran-4-yl}imidazo[1,2-b][1,2,4]triazin-6-yl)methyl]-4-methyl-1,2,5-oxadiazole-3-carboxamide;
N—[(S)-(4,4-Difluorocyclohexyl)(3-{4-[(4R)-2-oxo-4-(trifluoromethyl)imidazolidin-1-yl]-tetrahydropyran-4-yl}imidazo[1,2-b][1,2,4]triazin-6-yl)methyl]-4-methyl-1,2,5-oxadiazole-3-carboxamide;
tert-Butyl 4-(6-{(S)-(4,4-difluorocyclohexyl)[(4-methyl-1,2,5-oxadiazole-3-carbonyl)-amino]methyl}imidazo[1,2-b][1,2,4]triazin-3-yl)-4-[2-oxo-4-(trifluoromethyl)-imidazolidin-1-yl]piperidine-1-carboxylate;
N—[(S)-(4,4-Difluorocyclohexyl)(3-{4-[2-oxo-4-(trifluoromethyl)imidazolidin-1-yl]-piperidin-4-yl}imidazo[1,2-b][1,2,4]triazin-6-yl)methyl]-4-methyl-1,2,5-oxadiazole-3-carboxamide;
N—[(S)-(4,4-Difluorocyclohexyl)(3-{3-[(4S)-2-oxo-4-(trifluoromethyl)imidazolidin-1-yl]-oxetan-3-yl}imidazo[1,2-b][1,2,4]triazin-6-yl)methyl]-4-methyl-1,2,5-oxadiazole-3-carboxamide;
N—[(S)-(4,4-Difluorocyclohexyl)(3-{3-[(4R)-2-oxo-4-(trifluoromethyl)imidazolidin-1-yl]-oxetan-3-yl}imidazo[1,2-b][1,2,4]triazin-6-yl)methyl]-4-methyl-1,2,5-oxadiazole-3-carboxamide;
N—[(S)-(4,4-Difluorocyclohexyl)(3-{(3R)-3-[(4S)-2-oxo-4-(trifluoromethyl)imidazolidin-1-yl]tetrahydrofuran-3-yl}imidazo[1,2-b][1,2,4]triazin-6-yl)methyl]-4-methyl-1,2,5-oxadiazole-3-carboxamide;
N—[(S)-(4,4-Difluorocyclohexyl)(3-{(3S)-3-[(4S)-2-oxo-4-(trifluoromethyl)imidazolidin-1-yl]tetrahydrofuran-3-yl}imidazo[1,2-b][1,2,4]triazin-6-yl)methyl]-4-methyl-1,2,5-oxadiazole-3-carboxamide;
N—[(S)-(4,4-Difluorocyclohexyl)(3-{(3R)-3-[(4R)-2-oxo-4-(trifluoromethyl)imidazolidin-1-yl]tetrahydrofuran-3-yl}imidazo[1,2-b][1,2,4]triazin-6-yl)methyl]-4-methyl-1,2,5-oxadiazole-3-carboxamide;
N—[(S)-(4,4-Difluorocyclohexyl)(3-{(3S)-3-[(4R)-2-oxo-4-(trifluoromethyl)imidazolidin-1-yl]tetrahydrofuran-3-yl}imidazo[1,2-b][1,2,4]triazin-6-yl)methyl]-4-methyl-1,2,5-oxadiazole-3-carboxamide;
N—[(S)-(4,4-Difluorocyclohexyl)(3-{1-(2,2-difluoropropyl)-4-[(4S)-2-oxo-4-(trifluoro-methyl)imidazolidin-1-yl]piperidin-4-yl}imidazo[1,2-b][1,2,4]triazin-6-yl)methyl]-4-methyl-1,2,5-oxadiazole-3-carboxamide;
N—[(S)-(4,4-Difluorocyclohexyl)(3-{1-(2,2-difluoropropyl)-4-[(4R)-2-oxo-4-(trifluoro-methyl)imidazolidin-1-yl]piperidin-4-yl}imidazo[1,2-b][1,2,4]triazin-6-yl)methyl]-4-methyl-1,2,5-oxadiazole-3-carboxamide;
N—[(S)-(4,4-Difluorocyclohexyl)(3-{1-(2-fluoro-2-methylpropyl)-4-[(4S)-2-oxo-4-(trifluoromethyl)imidazolidin-1-yl]piperidin-4-yl}imidazo[1,2-b][1,2,4]triazin-6-yl)-methyl]-4-methyl-1,2,5-oxadiazole-3-carboxamide;
N—[(S)-(4,4-Difluorocyclohexyl)(3-{1-(2-fluoro-2-methylpropyl)-4-[(4R)-2-oxo-4-(trifluoromethyl)imidazolidin-1-yl]piperidin-4-yl}imidazo[1,2-b][1,2,4]triazin-6-yl)-methyl]-4-methyl-1,2,5-oxadiazole-3-carboxamide;
N—[(S)-(4,4-Difluorocyclohexyl)(3-{1-methyl-4-[(4S)-2-oxo-4-(trifluoromethyl)-imidazolidin-1-yl]piperidin-4-yl}imidazo[1,2-b][1,2,4]triazin-6-yl)methyl]-4-methyl-1,2,5-oxadiazole-3-carboxamide; or
N—[(S)-(4,4-Difluorocyclohexyl)(3-{1-methyl-4-[(4R)-2-oxo-4-(trifluoromethyl)-imidazolidin-1-yl]piperidin-4-yl}imidazo[1,2-b][1,2,4]triazin-6-yl)methyl]-4-methyl-1,2,5-oxadiazole-3-carboxamide; or
a pharmaceutically acceptable salt thereof.

11. (canceled)

12. (canceled)

13. (canceled)

14. A pharmaceutical composition comprising a compound of formula (I) as defined in claim 1 or an N-oxide thereof, or a pharmaceutically acceptable salt thereof, in association with a pharmaceutically acceptable carrier.

15. (canceled)

16. (canceled)

17. A method for the treatment and/or prevention of disorders for which the administration of a modulator of IL-17 function is indicated which comprises administering to a patient in need of such treatment an effective amount of a compound of formula (I) as defined in claim 1 or an N-oxide thereof, or a pharmaceutically acceptable salt thereof.

18. A method for the treatment and/or prevention of an inflammatory or autoimmune disorder, which comprises administering to a patient in need of such treatment an effective amount of a compound of formula (I) as defined in claim 1 or an N-oxide thereof, or a pharmaceutically acceptable salt thereof.

19. A compound as claimed in claim 3 wherein R¹ represents hydrogen.

20. A compound as claimed in claim 3 wherein R⁶ represents heteroaryl, which group may be optionally substituted by one or more substituents.

21. A compound as claimed in claim 4 wherein R⁶ represents heteroaryl, which group may be optionally substituted by one or more substituents.

22. A compound as claimed in claim 3 wherein ring A represents cyclopropyl, cyclobutyl, cyclopentyl, cyclohexyl, oxetanyl, tetrahydrofuranyl, pyrrolidinyl, tetrahydropyranyl or piperidinyl, any of which groups may be unsubstituted, or substituted by one, two or three substituents independently selected from halogen, cyano, C₁₋₆ alkyl, fluoro(C₁₋₆)alkyl, difluoro(C₁₋₆)alkyl, trifluoro(C₁₋₆)alkyl, hydroxy, C₁₋₆ alkoxy, C₁₋₆ alkylthio, C₁₋₆ alkylsulfinyl, C₁₋₆ alkylsulfonyl, C₂₋₆ alkylcarbonyl, C₂₋₆ alkoxycarbonyl, amino, C₁₋₆ alkylamino and di(C₁₋₆)alkylamino.

23. A compound as claimed in claim 22 wherein ring A represents cyclopropyl, cyclobutyl, oxetanyl, tetrahydrofuranyl, tetrahydropyranyl or piperidinyl, any of which groups may be unsubstituted, or substituted by one or two substituents independently selected from halogen, C₁₋₆ alkyl, fluoro(C₁₋₆)alkyl, difluoro(C₁₋₆)alkyl and C₂₋₆ alkoxy-carbonyl.

24. A compound as claimed in claim 6 wherein ring A represents cyclopropyl, cyclobutyl, cyclopentyl, cyclohexyl, oxetanyl, tetrahydrofuranyl, pyrrolidinyl, tetrahydropyranyl or piperidinyl, any of which groups may be unsubstituted, or substituted by one, two or three substituents independently selected from halogen, cyano, C₁₋₆ alkyl, fluoro(C₁₋₆)alkyl, difluoro(C₁₋₆)alkyl, trifluoro(C₁₋₆)alkyl, hydroxy, C₁₋₆ alkoxy, C₁₋₆ alkylthio, C₁₋₆ alkylsulfinyl, C₁₋₆ alkylsulfonyl, C₂₋₆ alkylcarbonyl, C₂₋₆ alkoxycarbonyl, amino, C₁₋₆ alkylamino and di(C₁₋₆)alkylamino.

25. A compound as claimed in claim 24 wherein ring A represents cyclopropyl, cyclobutyl, oxetanyl, tetrahydrofuranyl, tetrahydropyranyl or piperidinyl, any of which groups may be unsubstituted, or substituted by one or two substituents independently selected from halogen, C₁₋₆ alkyl, fluoro(C₁₋₆)alkyl, difluoro(C₁₋₆)alkyl and C₂₋₆ alkoxy-carbonyl.
